SEQUENCE LISTING (9 sequences)

SEQ ID NO: 1 - protein, 399 aa, Homo sapiens
MAFWGWRAAAALRLWGRVVERVEAGGGVGPFQACGCRLVLGGRDDVSAGLRGSHGARGEPLDPARPLQRPPRPEVPRAFRRQPRAAAPSFFFSSIKGGRRSISFSVGASSVVGSGGSSDKGKLSLQDVAELIRARACQRVVVMVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 2 - protein, 389 aa, Homo sapiens
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 3 - protein, 352 aa, Homo sapiens
MDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 4 - protein, 389 aa, Mus musculus
MAEPDPSDPLETQAGKVQEAQDSDSDTEGGATGGEAEMDFLRNLFTQTLGLGSQKERLLDELTLEGVTRYMQSERCRKVICLVGAGISTSAGIPDFRSPSTGLYANLEKYHLPYPEAIFEISYFKKHPEPFFALAKELYPGQFKPTICHYFIRLLKEKGLLLRCYTQNIDTLERVAGLEPQDLVEAHGTFYTSHCVNTSCRKEYTMGWMKEKIFSEATPRCEQCQSVVKPDIVFFGENLPPRFFSCMQSDFSKVDLLIIMGTSLQVQPFASLISKAPLATPRLLINKEKTGQTDPFLGMMMGLGGGMDFDSKKAYRDVAWLGDCDQGCLALADLLGWKKELEDLVRREHANIDAQSGSQAPNPSTTISPGKSPPPAKEAARTKEKEEQQ

SEQ ID NO: 5 - protein, 357 aa, Saccharomyces cerevisiae
MSVSTASTEMSVRKIAAHMKSNPNAKVIFMVGAGISTSCGIPDFRSPGTGLYHNLARLKLPYPEAVFDVDFFQSDPLPFYTLAKELYPGNFRPSKFHYLLKLFQDKDVLKRVYTQNIDTLERQAGVKDDLIIEAHGSFAHCHCIGCGKVYPPQVFKSKLAEHPIKDFVKCDVCGELVKPAIVFFGEDLPDSFSETWLNDSEWLREKITTSGKHPQQPLVIVVGTSLAVYPFASLPEEIPRKVKRVLCNLETVGDFKANKRPTDLIVHQYSDEFAEQLVEELGWQEDFEKILTAQGGMGDNSKEQLLEIVHDLENLSLDQSEHESADKKDKKLQRLNGHDSDEDGASNSSSSQKAAKE

SEQ ID NO: 6 - protein, 355 aa, Drosophila melanogaster
MDKVRRFFANTLHLGGSSDAKEEVKVEKVIPDLSFDGFAEHWRVHGFRKIVTMVGAGISTSAGIPDFRSPGSGLYSNLKKYELPHPTAIFDLDYFEKNPAPFFALAKELYPGSFIPTPAHYFIRLLNDKGLLQRHYTQNIDTLDRLTGLPEDKIIEAHGSFHTNHCIKCRKEYDMDWMKAEIFADRLPKCQKCQGVVKPDIVFFGENLPKRFYSSPEEDFQDCDLLIIMGTSLEVQPFASLVWRPGPRCIRLLINRDAVGQASCVLFMDPNTRSLLFDKPNNTRDVAFLGDCDAGVMALAKALGWDQELQQLITSERKKLSGSQNSEELQQGKEKPQSDPDKMTSGDRDKKDASL

SEQ ID NO: 7 - protein, 257 aa, Mus musculus
MVGAGISTPSGIPDFRSPGSGLYSNLQQYDIPYPEAIFELGFFFHNPKPFFMLAKELYPGHYRPNVTHYFLRLLHDKELLLRLYTQNIDGLERASGIPASKLVEAHGTFVTATCTVCRRSFPGEDIWADVMADRVPRCAVCTGVVKPDIVFFGEQLPARFLLHMADFALADLLLILGTSLEVEPFASLSEAVQKSVPRLLINRDLVGPFVLSPRRKDVVQLGDVVHGVERLVDLLGWTQELLDLMQRERGKLDGQDR

SEQ ID NO: 8 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 9 - protein, 652 aa, Mus musculus
MAEAPQVVETDPDFEPLPRQRSCTWPLPRPEFNQSNSTTSSPAPSGGAAANPDAAASLASASAVSTDFMSNLSLLEESEDFARAPGCVAVAAAAAASRGLCGDFQGPEAGCVHPAPPQPPPTGPLSQPPPVPPSAAAAAGPLAGQPRKTSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRGRAAKKKASLQSGQEGPGDSPGSQFSKWPASPGSHSNDDFDNWSTFRPRTSSNASTISGRLSPIMTEQDDLGDGDVHSLVYPPSAAKMASTLPSLSEISNPENMENLLDNLNLLSSPTSLTVSTQSSPGSMMQQTPCYSFAPPNTSLNSPSPNYSKYTYGQSSMSPLPQMPMQTLQDSKSSYGGLNQYNCAPGLLKELLTSDSPPHNDIMSPVDPGVAQPNSRVLGQNVMMGPNSVMPAYGSQASHNKMMNPSSHTHPGHAQQTASVNGRTLPHVVNTMPHTSAMNRLTPVKTPLQVPLSHPMQMSALGSYSSVSSCNGYGRMGVLHQEKLPSDLDGMFIERLDCDMESIIRNDLMDGDTLDFNFDNVLPNQSFPHSVKTTTHSWVSG

(19) United States
(12) Patent Application Publication
(10) Pub. No.: US 2009/0215681 A1
(43) Pub. Date: Aug. 27, 2009

(54) Methods of Diagnosis and Treatment of Metabolic Disorders

(75) Inventors: Ronald Kahn, West Newton, MA (US); Enxuan Jing, Chestnut Hill, MA (US)

Correspondence Address: CLARK & ELBING LLP, 101 FEDERAL STREET, BOSTON, MA, 02110, US

(73) Assignee: Joslin Diabetes Center

(21) Appl. No.: 11/883,867

(22) PCT Filed: Feb. 15, 2006
(86) PCT No.: PCT/US06/05493

§ 371 Date: Jun. 11, 2008

Related U.S. Application Data
(60) Provisional application No. 60/652,934, filed on Feb. 15, 2005.
(60) Provisional application No. 60/687,215, filed on Jun. 3, 2005.

(51) Int. Cl. A61K 38/16; C12Q 1/68; G01N 33/53; G01N 33/566; C40B 30/04; A61K 31/7088; A61K 31/7105; A61P 3/10

(52) U.S. Cl. 514/12; 435/6; 435/7.1; 436/501; 506/9

(57) ABSTRACT

The invention features diagnostic methods for metabolic disorders (e.g., diabetes and obesity), methods for screening for compounds useful in the treatment of metabolic disorders, and methods for treatment of metabolic disorders that involve sirtuin2 or sirtuin3 nucleic acids or proteins or their agonists or antagonists.

### CROSS-REFERENCE TO RELATED APPLICATIONS

This application claims benefit of U.S. Provisional Application Nos. 60/687,215, filed Jun. 3, 2005, and 60/652,934, filed Feb. 15, 2005, each of which is hereby incorporated by reference.

### STATEMENT AS TO FEDERALLY SPONSORED RESEARCH

The present research was supported by a grant from the National Institutes of Health (Numbers DK36836-15, DK33201, and DK45935). The U.S. Government may therefore have certain rights to this invention.

### BACKGROUND OF THE INVENTION

The invention relates to field of metabolic disorders, methods of diagnosing and treating such disorders, and screening methods for identification of compounds useful in treating metabolic disorders.

As the levels of blood glucose rise postprandially, insulin is secreted and stimulates cells of the peripheral tissues (skeletal muscles and fat) to actively take up glucose from the blood as a source of energy. Loss of glucose homeostasis as a result of faulty insulin secretion or action typically results in metabolic disorders such as diabetes, which may be co-triggered or further exacerbated by obesity. Because these conditions are often fatal, strategies to restore adequate glucose clearance from the bloodstream are required.

Diabetes mellitus, which results from a loss of insulin action on peripheral tissues, is a complex metabolic disorder accompanied by alterations in cellular physiology, metabolism, and gene expression and is one of the most common causes of morbidity and mortality in westernized countries (Skyler and Oddo, (2002) Diabetes Metab. Res. Rev. 18 Suppl 3, S21-S26). Although diabetes may arise secondary to any condition that causes extensive damage to the pancreas (e.g., pancreatitis, tumors, administration of certain drugs such as corticosteroids or pentamidine, iron overload (e.g., hemochromatosis), acquired or genetic endocrinopathies, and surgical excision), the most common forms of diabetes typically arise from primary disorders of the insulin signaling system. There are two major types of diabetes, namely type 1 diabetes (also known as insulin dependent diabetes (IDDM)) and type 2 diabetes (also known as insulin independent or non-insulin dependent diabetes (NIDDM)), which share common long-term complications in spite of their different pathogenic mechanisms.

Type 1 diabetes, which accounts for approximately 10% of all cases of primary diabetes, is an organ-specific autoimmune disease characterized by the extensive destruction of the insulin-producing beta cells of the pancreas. The consequent reduction in insulin production inevitably leads to the deregulation of glucose metabolism. While the administration of insulin provides significant benefits to patients suffering from this condition, the short serum half-life of insulin is a major impediment to the maintenance of normoglycemia. An alternative treatment is islet transplantation, but this strategy has been associated with limited success.

Type 2 diabetes, which affects a larger proportion of the population, is characterized by a deregulation in the secretion of insulin and/or a decreased response of peripheral tissues to insulin, i.e., insulin resistance. While the pathogenesis of type 2 diabetes remains unclear, epidemiologic studies suggest that this form of diabetes results from a collection of multiple genetic defects or polymorphisms, each contributing its own predisposing risks and modified by environmental factors, including excess weight, diet, inactivity, drugs, and excess alcohol consumption. Although various therapeutic treatments are available for the management of type 2 diabetes, they are associated with various debilitating side effects. Accordingly, patients diagnosed with or at risk of having type 2 diabetes are often advised to adopt a healthier lifestyle, including loss of weight, change in diet, exercise, and moderate alcohol intake. Such lifestyle changes, however, are not sufficient to reverse the vascular and organ damages caused by diabetes.

Given that the strategies currently available for the management of metabolic disorders such as diabetes and obesity are suboptimal, there is a compelling need for treatments that are more effective and are not associated with such debilitating side-effects.

### SUMMARY OF THE INVENTION

In one embodiment, the invention provides a method of diagnosing a metabolic disorder (e.g., diabetes), or a propensity thereto, in a subject (e.g., a human). The method includes analyzing the level of sirtuin3 expression or activity in a sample isolated from the subject, where a decreased level of sirtuin3 expression or activity in the sample relative to the level in a control sample indicates that the subject has the metabolic disorder, or a propensity thereto. The analyzing may include measuring the amount of sirtuin3 RNA or protein in the sample or measuring the histone deacetylase activity of sirtuin3 in the sample.

In another embodiment, the invention provides a method of identifying a candidate compound useful for treating a metabolic disorder (e.g., diabetes) in a subject. The method includes contacting a sirtuin3 protein (e.g., human sirtuin3 protein) with a compound (e.g., a compound from a chemical library); and measuring the activity of the sirtuin3, where an increase in sirtuin3 activity in the presence of the compound relative to sirtuin3 activity in the absence of the compound identifies the compound as a candidate compound for treating a metabolic disorder in a subject. The method may be performed in vivo (for example, in a cell or animal) or in vitro.

In a related embodiment, the invention provides another method of identifying a candidate compound useful for treating a metabolic disorder (e.g., diabetes) in a subject. The method includes contacting a sirtuin3 protein (e.g., human sirtuin3 protein) with a compound (e.g., a compound from a chemical library); and measuring the binding of the compound to sirtuin3, where specific binding of the compound to the sirtuin3 protein identifies the compound as a candidate compound for treating a metabolic disorder in a subject.

In another related embodiment, the invention provides a third method for identifying a candidate compound useful for treating a metabolic disorder (e.g., diabetes) in a subject. The method includes contacting a cell or cell extract including a polynucleotide encoding sirtuin3 (e.g., human sirtuin3) with a compound (e.g., a compound from a chemical library); and measuring the level of sirtuin3 expression in the cell or cell extract, where an increased level of sirtuin3 expression in the presence of the compound relative to the level in the absence of the compound identifies the compound as a candidate compound for treating a metabolic disorder in a subject.

The invention further provides a method of treating a metabolic disorder (e.g., diabetes) in a subject (e.g., a human). The method includes administering to the subject a composition that increases sirtuin3 expression or activity. The composition may include the sirtuin3 protein or a polynucleotide encoding the sirtuin3 protein.

In a related embodiment, the invention provides a kit for treating a metabolic disorder in a subject. The kit includes a composition that increases sirtuin3 expression or activity; and instructions for administering the composition to a subject with a metabolic disorder.

The present invention also provides methods that relate to applicants' newly discovered role of sirtuin2 in metabolic disorders. In a first embodiment, the invention provides a method of diagnosing a metabolic disorder (e.g., obesity), or a propensity thereto, in a subject (e.g., a human). The method includes analyzing the level of sirtuin2 expression or activity in a sample isolated from the subject, where an increased level of sirtuin2 expression or activity in the sample relative to the level in a control sample indicates that the subject has the metabolic disorder, or a propensity thereto. The analyzing may include measuring in the sample the amount of sirtuin2 RNA or protein, the histone deacetylase activity of sirtuin2, the deacetylation of Foxo1 by sirtuin2, or the binding of sirtuin2 to Foxo1.

In another embodiment, the invention provides a method of identifying a candidate compound useful for treating a metabolic disorder (e.g., obesity) in a subject. The method includes contacting a sirtuin2 protein (e.g., human sirtuin2 protein) with a compound (e.g., a compound selected from a chemical library); and measuring the activity of the sirtuin2 (e.g., binding to or deacetylation of Foxo1), where a decrease in sirtuin2 activity in the presence of the compound relative to sirtuin2 activity in the absence of the compound identifies the compound as a candidate compound for treating a metabolic disorder in a subject. The method may be performed in vivo (for example, in a cell or animal) or in vitro.

In another embodiment, the invention provides a method of identifying a candidate compound useful for treating a metabolic disorder (e.g., obesity) in a subject. The method includes contacting a sirtuin2 protein (e.g., human sirtuin2 protein) with a compound (e.g., a compound selected from a chemical library); and measuring the binding of the compound to sirtuin2, where specific binding of the compound to the sirtuin2 protein identifies the compound as a candidate compound for treating a metabolic disorder in a subject.

In a related embodiment, the invention provides another method for identifying a candidate compound useful for treating a metabolic disorder (e.g., obesity) in a subject. The method includes contacting a cell or cell extract including a polynucleotide encoding sirtuin2 (e.g., human sirtuin2) with a compound (e.g., a compound selected from a chemical library); and measuring the level of sirtuin2 expression in the cell or cell extract, where a decreased level of sirtuin2 expression in the presence of the compound relative to the level in the absence of the compound identifies the compound as a candidate compound for treating a metabolic disorder in a subject.

In another embodiment, the invention provides a method of treating a metabolic disorder (e.g., obesity) in a subject (e.g., a human). The method includes administering to the subject a composition that decreases sirtuin2 expression or activity, for example, a histone deacetylase inhibitor, dominant negative sirtuin2 (e.g., human H232Y sirtuin2), or an antibody that specifically binds sirtuin2, or a sirtuin2-binding fragment thereof. The decreased sirtuin2 activity includes binding to or deacetylation of Foxo1. Alternatively, the method may involve administering a nucleic acid that acts by RNA interference to block the mRNA coding for the sirtuin2 protein.

In a related embodiment, the invention provides a kit for treating a subject with a metabolic disorder. The kit includes a composition that decreases sirtuin2 expression or activity (e.g., binding to or deacetylation of Foxo1); and instructions for administering the composition to a subject with a metabolic disorder.

By “sirtuin3” is meant a polypeptide with at least 30%, 40%, 50%, 60%, 70%, 80%, 90%, 95%, or 99% sequence identity to SEQ ID NO:1 or a fragment thereof (FIG. 1) or a polypeptide encoded by a polynucleotide that hybridizes to a polynucleotide encoding SEQ ID NO:1 or a fragment thereof.

By “sirtuin2” is meant a polypeptide with at least 30%, 40%, 50%, 60%, 70%, 80%, 90%, 95%, or 99% sequence identity to SEQ ID NO:2, SEQ ID NO:3, or a fragment thereof (FIG. 1) or a polypeptide encoded by a polynucleotide that hybridizes to a polynucleotide encoding SEQ ID NO:2, SEQ ID NO:3, or a fragment thereof.

By “Foxo1” is meant a polypeptide with at least 30%, 40%, 50%, 60%, 70%, 80%, 90%, 95%, or 99% sequence identity to SEQ ID NO:8, or a fragment thereof, or a polypeptide encoded by a polynucleotide that hybridizes to a polynucleotide encoding SEQ ID NO:8, or a fragment thereof.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “hybridize” is meant pair to form a double-stranded complex containing complementary paired nucleic acid sequences, or portions thereof, under various conditions of stringency. (See, e.g., Wahl and Berger, (1987) Methods Enzymol. 152, 399-407; Kimmel, (1987) Methods Enzymol. 152, 507-511). For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180 (1977)); Grunstein and Hogness ((1975) Proc. Natl. Acad. Sci. USA 72, 3961); Ausubel et al. (Current Protocols in Molecular Biology, Wiley Interscience, New York (2001)); Berger and Kimmel (Guide to Molecular Cloning Techniques, Academic Press, New York, (1987)); and Sambrook et al. (Molecular Cloning: A Laboratory Manual, Cold Spring Harbor Laboratory Press, New York). Preferably, hybridization occurs under physiological conditions. Typically, complementary nucleobases hybridize via hydrogen bonding, which may be Watson-Crick, Hoogsteen, or reversed Hoogsteen hydrogen bonding, between complementary nucleobases. For example, adenine and thymine are complementary nucleobases that pair through the formation of hydrogen bonds.

By “fragment” is meant a chain of at least 4, 5, 6, 8, 10, 15, 20, or 25 amino acids or nucleotides which comprises any portion of a larger protein or polynucleotide.

By “biological sample” or “sample” is meant a sample obtained from an organism or from components (e.g., cells) of an organism. The sample may be of any biological tissue or fluid. Frequently the sample will be a “clinical sample” which is a sample derived from a subject. Such samples include, but are not limited to, sputum, blood, blood cells (e.g., white cells), tissue or fine needle biopsy samples, urine, peritoneal fluid, and pleural fluid, or cells. Biological samples may also include sections of tissues such as frozen sections taken for histological purposes.

By “subject” is meant either a human or non-human animal.

“Treating” a disease or condition in a subject or “treating” a subject having a disease or condition refers to subjecting the individual to a pharmaceutical treatment, e.g., the administration of a drug, such that at least one symptom of the disease or condition is decreased, stabilized, or prevented.

By “specifically binds” or “specific binding” is meant a compound or antibody which recognizes and binds a polypeptide of the invention but which does not substantially recognize and bind other molecules in a sample, for example, a biological sample, which naturally includes a polypeptide of the invention.

By “decrease in the level of expression or activity” of a gene is meant a reduction in protein or nucleic acid level or activity in a cell, a cell extract, or a cell supernatant. For example, such a decrease may be due to reduced RNA stability, transcription, or translation, increased protein degradation, or RNA interference. Preferably, this decrease is at least 5%, 10%, 25%, 50%, 75%, 80%, or even 90% of the level of expression or activity under control conditions.

By “increase in the expression or activity” of a gene or protein is meant a positive change in protein or nucleic acid level or activity in a cell, a cell extract, or a cell supernatant. For example, such a increase may be due to increased RNA stability, transcription, or translation, or decreased protein degradation. Preferably, this increase is at least 5%, 10%, 25%, 50%, 75%, 80%, 100%, 200%, or even 500% or more over the level of expression or activity under control conditions.

By a “compound,” “candidate compound,” or “factor” is meant a chemical, be it naturally-occurring or artificially-derived. Compounds may include, for example, peptides, polypeptides, synthetic organic molecules, naturally-occurring organic molecules, nucleic acid molecules, and components or combinations thereof.

By an “HDAC inhibitor” is meant any compound that reduces the activity of a histone deacetylase. Preferable HDAC inhibitors reduce activity by at least 5%, 10%, 25%, 50%, 75%, 80%, or even 100% as compared to an untreated control. Preferably, the HDAC inhibitor is specific for a Class III HDAC, and most preferably is specific or selective for sirtuin2.

By a “metabolic disorder” is meant any pathological condition resulting from an alteration in a mammal's metabolism. Such disorders include those resulting from an alteration in glucose homeostasis resulting, for example, in hyperglycemia. According to this invention, an alteration in glucose level is typically a glucose level that is increased by at least 5%, 10%, 20%, 30%, 40%, 50%, 75%, 100%, 125%, 150%, 200%, or even 250% relative to such levels in a healthy individual under identical conditions. Metabolic disorders include, for example, obesity and diabetes (e.g., diabetes type I, diabetes type II, MODY diabetes, and gestational diabetes).

Other features and advantages of the invention will be apparent from the following Detailed Description, the drawings, and the claims.

### BRIEF DESCRIPTION OF THE DRAWINGS

FIG. 1 is a list of amino acid sequences including human sirtuin3 (SEQ ID NO: 1) and both isoforms of human sirtuin2 (SEQ ID NO:2 and SEQ ID NO:3).

FIG. 2 is a schematic diagram of the proposed link between diabetes-induced metabolic changes and the derepression/induction of ribosomal protein related genes by means of sir2 histone deacetylase.

FIGS. 3A and 3B are schematic drawings showing the experimental design. FIG. 3A shows MIRKO mice and their Lox control littermates were treated with either STZ or citrate buffer. The diabetic (blood sugar, >400 mg/dl) mice were either followed or treated with insulin (blood sugar, <200 mg/dl) (STZ-insulin group). FIG. 3B shows genes that are altered significantly in expression in the MIRKO, Lox-STZ, and MIRKO-STZ groups are shown in a Venn diagram.

FIGS. 4A and 4B are graphs showing insulin-regulated versus diabetes-regulated gene expression. FIG. 4A shows a comparison of gene expression in Lox-STZ diabetic and MIRKO mice. The log of the ratios of the expression (experimental group/control) of genes that are changed significantly in either MIRKO or the Lox-STZ when compared with the Lox control are plotted on a log scale (every 0.3 units on the scale equals a 2-fold change). This comparison separated the genes into four quadrants, each reflecting either a concordant or discordant regulation of the genes by the loss of insulin-receptor-mediated signaling and the diabetic state. The genes labeled A and D, for example, were altered in diabetes, but they were not altered by a pure loss of insulin action in the MIRKO mouse; in contrast, the genes labeled B and C were altered in the MIRKO mouse but not in STZ diabetes. FIG. 4B shows the log of the ratios of the expression (experimental group/control) of genes that are changed significantly in either the MIRKO or the MIRKO-STZ when compared with the Lox control. The diagonal black line indicates the line of unity.

FIG. 5 is a table of genes showing direct vs. indirect effect of insulin on gene regulation. Some of the genes shown in FIG. 4B are represented here. The GenBank accession numbers, functional categories, and protein product names of the genes are given in the first three columns. The fifth column represents the change seen in the muscle insulin receptor knockout (MIRKO) mouse for these genes, whereas the sixth column represents the calculated value from FIG. 4B (as described herein) that represents the indirect effect of the loss of insulin (i.e., the change due to metabolic alterations of diabetes effect).

FIG. 6 is a graph showing the “loss-of-insulin effect” and the calculated “diabetes effect” are shown for representative genes. The loss-of-insulin effect was calculated from the percentage of change in expression in the MIRKO as compared with the Lox controls. The diabetes effect was calculated as the difference between the percentage of change in the MIRKO-STZ and MIRKO when compared with the Lox controls.

FIGS. 7A and 7B are graphs showing contrasting patterns of diabetes- and insulin-regulated genes. FIG. 7A shows the ratios of the expressions (experimental/Lox control) of all of the genes of the electron-transport chain that were changed significantly in the diabetic groups. All of these genes are changed significantly in the diabetic groups (Lox-STZ and MIRKOSTZ) but not in the MIRKO group. With insulin treatment, all of these genes corrected toward the Lox control by >50% in the Lox-STZ-INS but not in the MIRKO-STZ-INS group. The indicated genes are subunits of the electron-transport chain complexes I-V (C-I-C-V). FIG. 7B shows the ratios of the expression (experimental/Lox-control) of the genes for carnitine palmitoyl CoA transferasel (CPT1), 33-S2 enoyl CoA hydratase, cAMP-dependent protein kinase, and ubiquitin-specific protease 2. All of these genes are changed significantly in the diabetic groups (Lox-STZ and MIRKO-STZ) but not in the MIRKO group. With insulin treatment, all of these genes corrected toward the control by >50% in the Lox-STZ-INS but not in the MIRKO-STZ-INS group.

FIGS. 8A-8C are graphs and images showing changes in sirtuin3 and sir2 with diabetes. FIG. 8A shows the mean transcript levels of sirtuin3 in skeletal muscle in the various metabolic groups, as detected by microarray analysis, are shown as a percentage of the level in the control group. FIG. 8B shows the bands for sir2 in the nuclear (N) and cytosolic (C) fractions from the hind-limb muscles of wild-type control and STZ-induced diabetic mice are shown on immunoblots. FIG. 8C shows the mean intensity of the nuclear and cytosolic fraction sir2 bands on immunoblotting from two control and two diabetic mice are shown. The total is the sum of the respective nuclear and cytosolic fractions. The levels are represented as a percentage of the mean total level in the control group.

FIG. 9 is a set of graphs showing the ratios of the expressions (experimental group/Lox-control) of eukaryotic translation initiation factor (eIF) 2b and eIF 4e-binding protein (eIF 4e-bp). These genes are significantly changed in the diabetic groups (Lox-STZ and MIRKO-STZ) but not in the MIRKO group. Their individual GenBank accession numbers are given in Tables 1-4 and FIG. 5.

FIG. 10 is a combination of a schematic diagram of an experiment and set of images showing that adenoviral gene transfer of Sirt2 into pluripotent C3H10 stem cells promotes adipogenesis. The first column of images shows the Sirt2-overexpressing and control cells at Day 0 (two days post-confluence). Cells at Day 6 untreated or treated with the MIX (combination of dexamethasone (dex), a glucocorticoid that induces preadipocyte differentiation; IBMX, a compound that inhibits cAMP degradation, thus induces cAMP sensitive gene expression and differentiation; and insulin (ins)) from Day 0 to Day 2 are shown in column two and column three images, indicating that in both cases Sirt2-overexpressing cells show enhanced differentiation over control cells. The fourth column shows cells treated with thiazolidinedione (TZD), a PPARγ agonist, from Day 0 to Day 6.

FIG. 11 is a set of images from an Oil Red 0 staining experiment performed similarly to the experiment of FIG. 10. GFP or Sirt2 transfected cells eight days post-confluence (Day 6) were analyzed using the Oil Red O stain following no induction of adipocyte differentiation (control), induction with MIX (Ins/Dex/IBMX), or induction with TZD. Cells transfected with sirtuin2 differentiate into adipocytes to a greater extent than control cells.

FIG. 12 is a schematic diagram showing temporal expression of major transcription factors during adipogenesis (Rangwala and Lazar, (2002) Annu. Rev. Nutr. 20, 535-559).

FIG. 13 is a set of graphs showing that Sirt2 overexpression promotes mRNA expression of different adipogenetic genes in 3T3 L1 cells, including PPARγ, C/EBPα, aP2, Glut4, and FAS.

FIG. 14 is a set of graphs showing activation of PPARγ2 and aP2 promoter by Sirt2.

FIG. 15 is a set of western blot images showing the effect of insulin concentration (0, 10 nm, 100 nm) on the expression of P-Akt, P-Erk, and P-p38 in 3T3 L1 CAR cells overexpressing and control cells not overexpressing Sirt2. Significant differences between the cells overexpressing Sirt2 and control are not observed.

FIG. 16 is a set of western blot images showing that Sirt2 overexpression promotes PPARγ expression but not C/EBPα in 3T3 L1 cells.

FIG. 17 is a set of western blot images showing the effect of Sirt2 overexpression on C/EBPα and Glut4 expression.

FIG. 18A is a schematic diagram of the constructs used for Sirt2 RNA interference (RNAi) experiments. FIG. 18B is a graph showing decreases in Sirt2 expression, but not Sirt1 or Sirt3 expression, upon treatment of C3H10 cells with two different Sirt2 RNAi constructs (S2-1 and S2-2 siRNA constructs target exon 4 and exon 9 of mouse sirtuin2, respectively), as compared to cells receiving a GFP RNAi construct. FIG. 18C is a photomicrograph showing decreased C3H10 cell line adipogenesis upon treatment with MIX in cells containing a Sirt2 RNAi construct as compared to control cells.

FIG. 19A is a depiction of acetylation and phosphorylation sites of mouse Foxo1 (SEQ ID NO:9), a transcription factor regulated by its acetylation state. FIG. 19B is a schematic diagram showing that CBP (cAMP-response element-binding protein-binding protein) regulates Foxo1 activity by acetylating Foxo1, and that PKB (protein kinase B; Akt) phosphorylates the acetylated Foxo1.

FIGS. 20A and 20B are photographs of western blots. FIG. 20A shows lysates from 3T3L1 cells expressing either a GFP siRNA or a sirtuin2 siRNA. Both lysates show similar expression levels of Foxo1 and sirtuin1.

FIG. 20B shows western blots of immunoprecipitations performed using either anti-Ack (anti-acetylated lysine) or anti-IgG. These results indicate that decreasing sirtuin2 expression results in increased acetylation of Foxo1.

FIGS. 21A and 21B are photographs of western blots. FIG. 21A shows anti-Foxo1 western blots of cytosolic and nuclear fractions of cell lysates with either a GFP targeted siRNA or a sirtuin2 targeted siRNA. These results indicate that reduced sirtuin2 expression increases the amount of acetylated, cytosolic Foxo1, thereby implicating sirtuin2 in the cytosolic/nuclear translocation of Foxo1. FIG. 21B shows western blots for p-Akt, Akt, and p-Foxo1 in cells expressing GFP-targeted siRNA or sirtuin2-targeted siRNA at 0, 10, and 100 nmol concentrations. p-Foxo1 is increased in the sirtuin2 knockdown cells as compared to cells with the GFP-targeted siRNA, the latter of which also have considerable Foxo1 phosphorylation under insulin treatment.

FIGS. 22A and 22B are western blots showing that sirtuin2 interacts with Foxo1 in vitro. FIG. 22A shows that the starting materials of sirtuin2-FLAG lysate and control lysate contain similar amounts of Foxo1. FIG. 22B shows that Foxo1 appears on a western blot of an immunoprecipitation using anti-FLAG in the presence of sirtuin2-FLAG but not on a western blot in the absence of sirtuin2-FLAG, thereby indicating an interaction between sirtuin2 and Foxo1.

FIG. 23 is a series of western blots showing that sirtuin2 overexpression (right column; sirtuin2-FLAG construct) does not alter components of the insulin signaling pathway including P-Akt, P-Erk, and P-p38, at 0, 10, and 100 nmol insulin concentrations.

FIG. 24 is a set of graphs showing that reduction of sirtuin2 expression by RNAi results in increased mRNA expression of aP2, FAS, and Glut4 in 3T3L1 cells.

FIG. 25 is a set of graphs showing that reduction of sirtuin2 expression by RNAi results in increased mRNA expression of PPARγ, C/EBPα, and Pref-1, but not sirtuin1 in 3T3L1 cells.

FIG. 26 is a set of photographs of western blots showing increased protein expression of C/EBPβ, C/EBPα, PPARγ, and FAS in 3T3L1 cells with sirtuin2-targeted siRNA.

### DETAILED DESCRIPTION

The present invention includes methods for the diagnosis and treatment of metabolic disorders such as diabetes and obesity as well as methods for identifying compounds useful in the treatment of metabolic disorders. These methods utilize the identification of two proteins, sirtuin3 and sirtuin2, as playing key roles in the pathogenesis of these diseases, as outlined below.

The following examples are meant to illustrate the invention and should not be construed as limiting.

### Example 1

### Sirtuin3 and Diabetes

Changes in gene expression in diabetes (Yechoor et al., (2002) Proc. Natl. Acad. Sci. USA 99, 10587-10592; Sreekumar et al., (2002) Diabetes 51, 1913-1920; O'Brien and Granner, (1996) Physiol. Rev. 76, 1109-1161) may be the result of (i) direct effects of decreased insulin action via receptor-mediated signaling, and (ii) indirect effects secondary to the metabolic and humoral changes associated with the disease. While recent studies (Mootha et al., (2003) Nat. Genet. 34, 267-273; Patti et al., (2003) Proc. Natl. Acad. Sci. USA 100, 8466-8471) have demonstrated a coordinated dysregulation of several genes encoding components of mitochondrial electron-transport in muscle of individuals with impaired glucose tolerance or type 2 diabetes and their insulin-resistant relatives, it was not previously possible to determine whether these alterations represent a direct result of the loss of insulin signaling due to insulin resistance, are secondary to the abnormal metabolism in these conditions, or are primary genetically determined defects.

To dissect and quantitate these two separate effects, the skeletal muscle gene-expression profiles of muscle insulin receptor knockout (MIRKO) mice and their Lox controls in the basal, streptozotocin-induced diabetic, and insulin-treated diabetic states are compared. Pure deficiency of insulin action as present in the MIRKO mouse results in changes in the expression level of 130 genes, including downregulation of NSF (N-ethylmaleimide-sensitive fusion protein), VAMP-2 (vesicle-associated membrane protein 2), stearoyl CoA desaturase 1, and cAMP-specific phosphodiesterase 4B, and upregulation of signaling-related genes (e.g., Akt2 and the fatty-acid transporter CD36). In diabetes, alterations in expression of about 500 genes can be observed, including a highly coordinated downregulation of genes of the mitochondrial electron-transport chain and one of the mammalian homologues of the histone deacetylase Sir2, sirtuin3, which has been implicated in a link between nutrition and longevity. Knowledge of these pathways provides insight into the complex mechanisms of transcriptional control in diabetes and provides potential therapeutic targets.

The creation of targeted genetic models in mice, such as the muscle insulin receptor knockout (MIRKO) mice, in which there is a complete absence of the insulin-receptor signaling in skeletal muscle but normal insulin and glucose levels (Bruning et al., (1998) Mol. Cell. 2, 559-569; Wojtaszewski et al., (1999) J. Clin. Invest. 104, 1257-1264), allows the use of genetics to separate the direct and indirect effects of insulin action in higher organisms. By comparing skeletal muscle gene-expression profiles from MIRKO mice and control mice under three different metabolic conditions (in the basal state, after streptozotocin (STZ)-induced diabetes, and after STZ-induced diabetes rendered euglycemic with insulin treatment), the following three issues can be addressed: (i) determination of the direct effect of the loss of insulin signaling on gene expression in skeletal muscle; (ii) determination of the contribution of the metabolic and other changes that accompany diabetes to induce indirect changes in gene expression; (iii) determination of how these pathways are regulated and implicated in the pathophysiology of diabetes. The results presented herein elucidate the genetic heterogeneity of diabetes and define targets (e.g., human sirtuin3) for therapy.

It can be challenging to define precise factors that regulate gene expression in vivo. Hormones, for example, produce many metabolic effects, any of which can secondarily alter gene expression. Additionally, small changes in gene expression can lead to cascading and amplifying effects on protein expression and metabolic pathways. In the case of insulin deficiency, changes in the levels of, for example, glucose, lipid and protein metabolites, other hormones, and ion flux, can regulate gene expression beyond the direct effects of the hormone. Here, comparison of MIRKO mice with STZ diabetes and control mice indicates that direct insulin action has a role in maintaining the basal expression levels of only a relatively modest subset (˜1%) of genes studied as compared with the larger number (˜4%) of genes studied that are altered in diabetes. One example of diabetes-mediated, rather than insulin-mediated, regulation is the nuclear encoded subunits of the mitochondrial electron-transport chain. Expression in the basal state (even in the absence of insulin action) is normal, whereas expression of 12 components of this system is decreased in diabetes. In the basal state, there is a lack of dependence on insulin action, but insulin receptor-mediated signaling is required to reverse the effects induced by diabetes. A converse pattern of upregulation occurs with other genes. This pattern of regulation suggests that the metabolic changes in diabetes may induce a repressor of gene expression that downregulates a family of genes (or, conversely, an activator that upregulates a family of genes), which has its own expression suppressed by direct insulin action. Thus, there is no effect of an isolated loss of insulin action in the MIRKO mouse in the basal state; however, when diabetes occurs and the repressor or activator is expressed, the presence of an intact insulin-signaling system is needed to restore normal expression.

These findings are particularly relevant to observations of coordinated downregulation of genes (Sreekumar et al., (2002) Diabetes 51, 1913-1920, Mootha et al., (2003) Nat. Genet. 34, 267-273, Patti et al., (2003) Proc. Natl. Acad. Sci. USA 100, 8466-8471) and decreased activity (Tsukiyama-Kohara et al., (2001) Nat. Med. 7, 1128-1132) of the electron-transport chain in muscle of insulin-resistant individuals with diabetes and aging, respectively. Based the present study demonstrating no changes in these genes in the MIRKO mouse, changes observed in these human studies are likely not a result of insulin resistance but the result of either independent, primary genetic alterations or alterations secondary to the processes of altered metabolism associated with diabetes and aging.

Proteins that regulate these diabetes-related changes include DR1, HAT type B, and sirtuin3. DR1 is a 176-amino acid protein that interacts with the TATA box-binding protein (TBP) in a phosphorylation-dependent manner to repress both basal and activated levels of transcription (Inostroza et al., (1992) Cell 70, 477-489). DR1 is upregulated in the MIRKO mouse (indicating that it is under insulin control), and it is further upregulated in the diabetic state. In addition, there is downregulation of HAT type B and sirtuin3, a homolog of the yeast Sir2, in STZ-induced diabetes. The Sir2 family of type III histone deacetylases is involved in NAD-dependent transcriptional repression and is thought to play an important role in the response to aging and caloric restriction (see below) (Blander and Guarente, (2004) Annu. Rev. Biochem. 73, 417-435). In the latter case, this function may be further modified by interactions at the biological level.

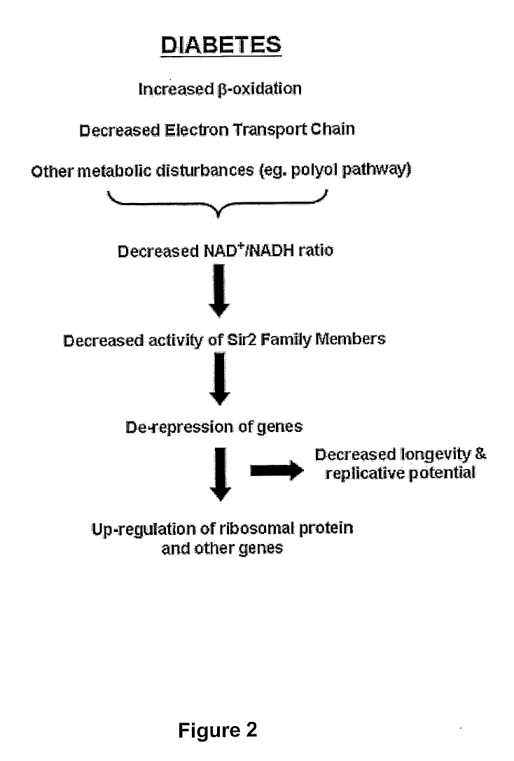

For example, a major portion of intracellular NADH, which is normally generated by the oxidative metabolism of glucose and fatty acids, is converted to NAD with a simultaneous generation of ATP by the electron-transport chain. Thus, a decrease in expression or activity of the electron-transport chain subunits seen in diabetes (Mootha et al., (2003) Nat. Genet. 34, 267-273; Patti et al., (2003) Proc. Natl. Acad. Sci. USA 100, 8466-8471) or aging (Petersen et al., (2003) Science 300, 1140-1142) may contribute to a decreased NAD⁺/NADH ratio. Indeed, studies have demonstrated reduced NAD⁺/NADH ratios in diabetes (Trueblood and Ramasamy, (1998) Am. J. Physiol. 275, H75-H83; Salceda et al., (1998) Neurochem. Res. 23, 893-897). Decreases in NAD⁺ may lead to a decrease in the activity of NAD⁺-dependent processes including the Sir2 NAD⁺-dependent histone deacetylases. Changes in Sir2-related activities may regulate gene expression for many ribosomal proteins (Smith and Boeke, (1997) Genes Dev. 11, 241-254; Straight et al., (1999) Cell 97, 245-256), and other proteins whose expression is altered in diabetes (Yechoor et al., supra). Sir2 family members also regulate muscle gene expression and differentiation possibly as a redox sensor in response to food intake and starvation (Fulco et al., (2003) Mol. Cell. 12, 51-62); an increase in Sir2 is associated with increased longevity induced by calorie restriction in C. elegans (Guarente and Kenyon, C. (2000) Nature 408, 255-262; Tissenbaum and Guarente, (2001) Nature 410, 227-230), yeast (Kaeberlein et al., (1999) Genes Dev. 13, 2570-2580; Lin et al., (2004) Genes Dev. 18, 12-16), flies (Rogina et al., (2002) Science 298, 1745), and mammalian cells (Cohen et al., (2004) Science 305, 390-392). A schematic model is shown in FIG. 2.

Sirtuin3 (human SEQ ID NO: 1; mouse SEQ ID NO:7), a member of this family, is also decreased both at the mRNA and protein level in diabetic mice. The exact role of sirtuin3 (SIRT3) in mammals is unknown, but it is preferentially localized in mitochondria (Onyango et al., (2002) Proc. Natl. Acad. Sci. USA 99, 13653-13658). Alterations in mitochondrial function (Kelley et al., (2002) Diabetes 51, 2944-2950) or in the mitochondrial electron-transport chain have been found in muscle of animal models of type 1 diabetes (Yechoor et al., supra) and humans with type 2 diabetes (Patti et al., (2003) Proc. Natl. Acad. Sci. USA 100, 8466-8471).

In summary, by using mouse genetics, genes that are regulated directly by insulin versus those that are regulated by the diabetic metabolic milieu have been defined in vivo. Furthermore, transcriptional regulatory mechanisms by which diabetes may coordinately regulate the expression of electron-transport chain subunits and fatty-acid metabolism-related genes have been identified. Knowledge of these pathways provides insight into mechanisms by which insulin and key metabolites control transcription, thus identifying possible targets for therapeutic intervention for metabolic disorder (e.g., diabetes), and suggesting mechanisms for the detrimental effect of diabetes on cellular longevity and replicative potential.

These experiments were carried out as follows.

### MIRKO Mice

Three groups of 6- to 8-week-old male MIRKO mice and their Lox controls were studied. One group of each genotype was given daily intraperitoneal (i.p.) injections of sodium citrate (pH 4.3) for 3 days (controls). A second group of each genotype was treated with an i.p. injection of 100 μg of STZ (Sigma) in sodium citrate (s.c.) per g of body weight for 3 consecutive days. When these mice achieved fed glucose levels of >400 mg/dl for 3 consecutive days, they were separated into two groups. One-half of these mice were not treated, and the other one-half were treated with s.c. insulin pellets (LinShin, Toronto, ON, Canada) to obtain fed glucose levels of <200 mg/dl for at least 3 consecutive days (Yechoor et al., supra). Thus, six experimental groups each consisting of at least six mice were created.

RNA was extracted from skeletal muscle, and two pools consisting of equal quantities of RNA from three mice within each group were created for each of the experimental groups. This pooled RNA and RNA from five or six individual mice in each group was used for hybridization to a total of seven or eight MG-U74A-v2 (Affymetrix, Santa Clara, Calif.) arrays per group. Data analysis, using three filters of significance to identify differentially expressed genes, was performed as described in Yechoor et al. (supra).

### Animals and Treatment Groups

Three groups of 6- to 8-week-old male MIRKO mice and their Lox/Lox controls were maintained on a 12-h light/12-h dark cycle and fed a standard mouse diet (9F 5020, Purina). One group of each was given daily i.p. injections of sodium citrate (pH 4.3) for 3 days (controls). A second group of each was treated with an i.p. injection of streptozotocin (Sigma), 100 μg/g body weight in sodium citrate for 3 consecutive days. When these mice achieved fed glucoses of >400 mg/dl for 3 consecutive days, they were separated into two groups. Half were not treated, and the half were treated with s.c. insulin pellets (LinShin, Toronto, ON, Canada), to obtain fed glucose <200 mg/dl for at least 3 consecutive days (unpublished data). Thus, six experimental groups each consisting of at least 6 mice were created. All mice were killed between 1:00 and 4:00 p.m., and hindlimb skeletal muscle was snap frozen in liquid nitrogen and stored at −80° C.

### RNA Extraction and Microarray Hybridization

Detailed methods have been described (Yechoor et al., supra). Briefly, RNA was extracted from muscle by using TRIzol (Invitrogen). Two pools consisting of equal quantities of RNA from three mice within each group were created for each of the experimental groups. This pooled RNA and RNA from five to six individual mice in each group were purified by using RNeasy (Qiagen, Valencia, Calif.), allowing for a total of seven to eight arrays per group. Because the animals were of mixed genetic background, this larger number of arrays minimized biological and methodological variability. Biotinylated cRNA was generated by using 25 μg of the RNA samples (Affymetrix, Santa Clara, Calif.) and quantitated after adjusting for carryover of residual RNA. We fragmented 15 μg of adjusted cRNA and hybridized it to MG-U74A-v2 arrays (Affymetrix) for 16 h, and it was then washed and scanned. Data were analyzed by using genechip microarray suite (version 5.0), genespring (version 4.1), and excel (Microsoft). Data analysis was performed as described by using three filters of significance (Yechoor et al., supra). First, all genes were excluded for which mean expression value was below the sum of the average background and the average standard difference threshold (SDT, four times scaled noise) in both control and the diabetic groups. Genes that passed the first filter were subjected to a second filter, which selected for genes with an absolute difference between the means of the control and experimental groups that was greater than the average SDT. The third filter considered only those genes that had a significance of P≦0.05, obtained with a two-tailed t test assuming unequal variance between groups. These genes were then labeled as being significantly changed between the control and the experimental groups. A gene was labeled as responsive to insulin treatment if the expression intensity of the gene in the insulin-treated group reverted toward the control by at least one-half of the expression difference between control and diabetic groups.

### Protein Extraction and Immunoblotting

Hindlimb muscles of two wild-type and two streptozotocin (STZ) diabetic mice were homogenized with a Polytron (Beckman Coulter) in-tissue lysis buffer (25 mM Tris.HCl pH 7.4, 2 mM sodium vanadate, 10 mM sodium fluoride, 10 mM sodium orthophosphate, 1 mM EDTA, 1 mM EGTA, 5 μg/ml leupeptin, 5 μg/ml aprotinin, 1 mM PMSF, 1% Nonidet P-40). The homogenate was centrifuged at 1,500 g for 10 min. The supernatant was then centrifuged at 30,000 g, and the resultant supernatant was used as the cytosolic protein extract after removing the upper fat layer. The pellet was washed with tissue lysis buffer with 25% glycerol and then lysed with the nuclear extraction buffer (nuclear wash buffer with 330 mM sodium chloride) by passing it through an 18G needle five times. This lysate was rotated at 4° C. for 20 min and then centrifuged at 14,000 g; the supernatant was collected as the nuclear/mitochondrial protein extract. Protein concentration was measured by using the Lowry method. Equal amounts of protein (1 mg) were immunoprecipitated at 4° C. for 12 h with anti-sir2 antibody (Zymed) and protein G Sepharose beads. After separation by SDS/PAGE, immunoprecipitates were subjected to western blotting with the same antibody and visualized by enhanced chemiluminescence (Pierce) and quantitated by using labworks (BioImaging Systems, Upland, Calif.).

Comparison of Gene Expression between MIRKO and Lox Control Mice

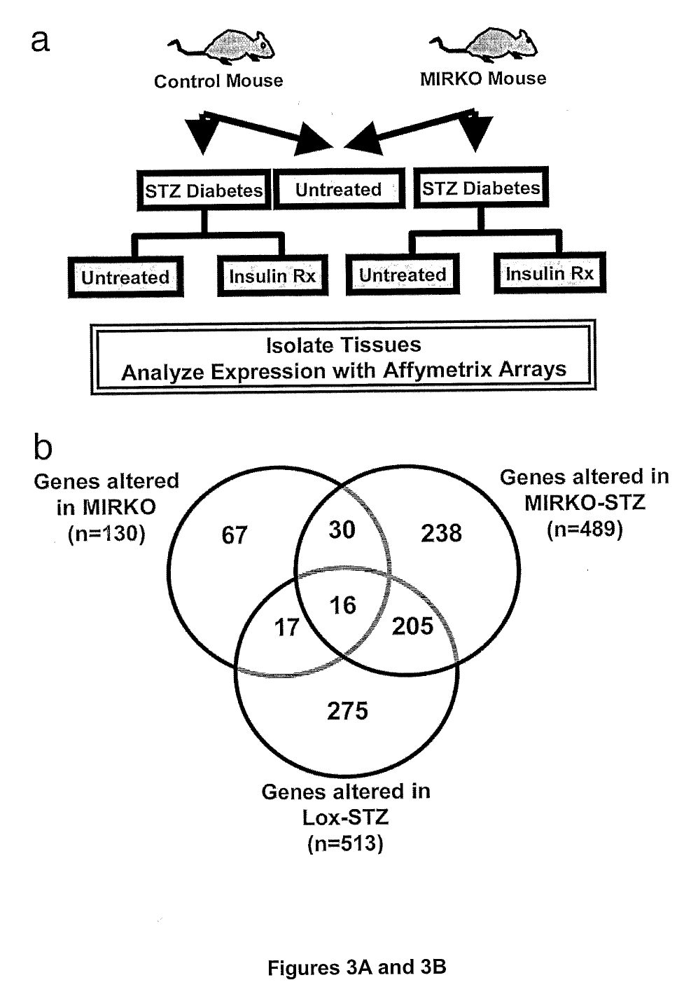

By using MG-U74Av2 oligonucleotide arrays, the expression of 12,488 genes and ESTs (hereafter referred to as genes) was analyzed in skeletal muscle derived from the following six groups of mice: MIRKO and Lox control in the basal state, MIRKO and Lox control in the STZ-induced diabetes state, and MIRKO and Lox control in STZ-induced diabetes made euglycemic by insulin-treatment states (FIG. 3A). Of the 12,488 genes represented on the chip, 130 genes were differentially expressed in muscle between MIRKO and control mice, thus defining the subset of genes regulated by insulin by means of insulin receptor-mediated signaling (FIG. 3B, and see Tables 1 and 2), and they were further grouped based on functional ontology. The following groups of genes were identified.

TABLE 1
Genes significantly downregulated in the MIRKO (isolated
loss of insulin receptor-mediated signaling) as compared
to the Lox-control group listed by functional class.

| | | |
|---|---|---|
| GenBank | | Fold change in |
| accession no. | Gene/protein | KO/Lox |

Metabolism-related

| | | |
|---|---|---|
| U79573 | Apolipoprotein A-I | 0.26 |
| AI853364 | Fatty-acid desaturase 1 (Stearoyl CoA desaturase 1) | 0.73 |
| AF058956 | Sucinate-coenzyme A ligase, GDP-forming, β subunit | 0.84 |

Signaling-related

| | | |
|---|---|---|
| AI840738 | Platelet-derived growth-factor receptor, α polypeptide | 0.43 |
| AA034874 | MAP/microtubule affinity-regulating kinase-like 1 | 0.44 |
| | (MARKL1) | |
| AF031939 | Placental growth factor | 0.57 |
| AI180687 | Phosphodiesterase 4B, cAMP-specific | 0.61 |
| AF031939 | RalBP1-associated Eps domain-containing protein | 0.61 |
| Y17345 | Protein tyrosine phosphatase IF-1 (LMW) | 0.66 |
| AI845103 | Podocalyxin-like | 0.89 |

Transcription/translation-related

| | | |
|---|---|---|
| AI425994 | Repilication intitiation origin | 0.59 |
| AW125218 | Histone Acetyltransferase Type B catalytic subunit | 0.59 |
| U66249 | Cut (Drosophila)-like 1 | 0.81 |

Transport/trafficking-related

| | | |
|---|---|---|
| U10120 | N-ethylmaleimide sensitive fusion protein | 0.49 |
| AF014461 | Exocyst component protein 70 kDa homolog | 0.72 |
| | (Saccharomyces cerevisiae) | |
| AI852124 | Brefeldin A-inhibited guanine nucleotide-exchange | 0.81 |
| | protein 1 (BIG1) | |
| AW122882 | Vesicle-associated membrane protein 2 | 0.82 |
| U31510 | ADP-ribosyltransferase 1 | 0.82 |

Membrane protein-related

| | | |
|---|---|---|
| X69966 | Cadherin 4 | 0.65 |
| X75927 | ATP-binding cassette 2 | 0.67 |
| U35741 | Thiosulfate sulfurtransferase, mitochondrial | 0.68 |
| AJ133427 | Olfactory receptor 37d | 0.73 |
| AB013729 | Semaphorin 6C | 0.74 |
| L48687 | Sodium channel, voltage-gated, type I, beta polypeptide | 0.76 |

Proteasome/protease-related

| | | |
|---|---|---|
| M75721 | Serine protease inhibitor 1-1 | 0.35 |
| M75718 | Serine protease inhibitor 1-4 | 0.40 |
| AV365271 | Neural precursor cell expressed, developmentally | 0.53 |
| | down-regulated gene 4a (ubiquitin-protein ligase) | |
| AI574278 | Insulin-degrading enzyme | 0.68 |
| AW125420 | Similar to Ubiquitin 2 | 0.68 |
| AI853269 | Proteasome (prosome, macropain) subunit, β type, 2 | 0.86 |

TABLE 2
Genes significantly upregulated in the MIRKO (isolated loss
of insulin receptor-mediated signaling) as compared with
the Lox-control group listed by functional class.

| | | |
|---|---|---|
| GenBank | | Fold change in |
| accession no. | Gene/protein | KO/Lox |

Metabolism-related

| | | |
|---|---|---|
| U20257 | Alcohol dehydrogenase 3 complex | 1.83 |
| M12330 | Ornithine decarboxylase, structural | 1.61 |
| AF032128 | Ornithine decarboxylase antizyme inhibitor | 1.51 |
| AJ006474 | Carbonic anhydrase 3 | 1.27 |
| Z49204 | Nicotinamide nucleotide transhydrogenase | 1.24 |
| M14220 | Glucose phosphate isomerase 1 complex | 1.22 |
| M15668 | Phosphoglycerate kinase 1 | 1.18 |

Signaling-related

| | | |
|---|---|---|
| AI882416 | Leptin (Ob) precursor | 1.38 |
| AF029982 | SERCA2 (ATPase, Ca++ transporting, cardiac | 1.34 |
| | muscle, slow twitch 2) | |
| AB008553 | CD36 antigen (collagen type I receptor, | 1.25 |
| | thrombospondin receptor)-like2 | |
| U22445 | Akt2 (thymoma viral proto-oncogene 2) | 1.21 |
| D29802 | Guanine nucleotide binding protein, β 2, related | 1.21 |
| | sequence 1 | |

Transcription/translation-related

| | | |
|---|---|---|
| AW122643 | DEAD BOX PROTEIN homolog (helicase) | 1.69 |
| AF051945 | Cardiac morphogenesis | 1.67 |
| U41626 | Split hand/foot deleted gene 1 | 1.26 |
| AW122452 | Speckle-type POZ protein | 1.23 |
| Z31555 | Chaperonin subunit 5 (epsilon) | 1.22 |
| M94087 | Activating transcription factor 4 | 1.21 |
| AI844751 | Similar to eukaryotic translation termination factor 1 | 1.17 |
| | (eRFI) | |
| AF100956 | BING1 | 1.17 |
| Z31399 | Chaperonin subunit 7 (eta) | 1.13 |

Transport/trafficking-related

| | | |
|---|---|---|
| K02236 | Metallothionein 2 | 2.23 |
| AF079901 | Golgi SNAP receptor complex member 1 | 1.55 |
| AI850000 | Dynein-associated protein homolog | 1.49 |
| M13018 | Cysteine-rich intestinal protein (Zinc transporter) | 1.23 |
| D87902 | ADP-ribosylation factor 5 | 1.17 |
| X52561 | Ferritin heavy chain | 1.13 |

Membrane protein-related

| | | |
|---|---|---|
| AI843029 | ATPase, H+ transporting, lysosomal (vacuolar proton | 1.3 |
| | pump), beta 56/58 kDa, isoform 2 | |
| X59047 | CD 81 antigen | 1.29 |

Structural protein-related

| | | |
|---|---|---|
| AJ223362 | Myosin heavy chain, cardiac muscle, fetal | 1.81 |
| D85923 | Myosin heavy chain 11, smooth muscle, | 1.74 |
| U04541 | Tropomyosin 5 | 1.7 |
| M29793 | Troponin C, cardiac/slow skeletal | 1.61 |
| D88791 | Cysteine-rich protein 3 | 1.6 |
| AJ011118 | Ankyrin repeat domain 2 (stretch responsive muscle) | 1.54 |
| X13297 | Actin, α 2, smooth muscle, aorta | 1.37 |
| AI462105 | Vinculin ortholog | 1.25 |
| AJ011118 | Ankyrin 1, erythroid | 1.18 |
| U38967 | Thymosin, β 4, X chromosome | 1.17 |
| L48989 | Troponin T3, skeletal, fast | 1.16 |
| AF093775 | Actinin α 3 | 1.14 |

Proteasome/Protease-related

| | | |
|---|---|---|
| M64086 | Serine protease inhibitor 2-2 | 1.6 |
| AV103587 | Protein C | 1.58 |
| AF026469 | Ubiquitous nuclear protein (ubiquitin-dependent | 1.25 |
| | protein degradation) | |
| U59807 | Cystatin B | 1.23 |
| U25844 | Serine protease inhibitor 3 | 1.17 |

### Signaling-Related Genes

cAMP-specific phosphodiesterase 4, which regulates many insulin- and glucagon-mediated pathways, including glycogen synthesis and glycogenolysis, was downregulated by 39% in MIRKO muscle. This result indicates that in the basal state, insulin would upregulate expression of this enzyme, resulting in a decrease in the level of cAMP (which normally opposes insulin action on carbohydrate metabolism). Expression of Akt2, which plays an important role in insulin-regulated metabolism and cell growth (Saltiel and Kahn, (2001) Nature 414, 799-806; Scheid and Woodgett, (2001) Nat. Rev. Mol. Cell. Biol. 2, 760-768), and SERCA2, which binds to IRS (insulin receptor substrate) proteins in an insulin-dependent manner (Algenstaedt et al., (1997) J. Biol. Chem. 272, 23696-23702), were increased in MIRKO (Table 2).

### Membrane- and Metabolism-Related Genes

CD36, a cell-surface fatty-acid transporter, whose deficiency has been associated with both insulin resistance (Aitman et al., (1999) Nat. Genet. 21, 76-83; Pravenec et al., (2001) Nat. Genet. 27, 156-158) and atherosclerosis (Febbraio et al., (2001) J. Clin. Invest. 108, 785-791; Nicholson et al., (2001) Ann. N.Y. Acad. Sci. 947, 224-228) was upregulated in MIRKO muscle, suggesting that insulin suppresses the expression of this protein. mRNA for ornithine decarboxylase and its antizyme inhibitor (which are both involved in synthesis of polyamines that have an important role in cell growth, replication, and the redox state) were upregulated by 61% and 51%, respectively, in MIRKO muscle, indicating that insulin signaling has a tonic inhibitory influence on expression and activity of ornithine decarboxylase in muscle, leading to an increase in its activity. Stearoyl CoA desaturase 1 (SCD-1), which catalyzes an important step in the biosynthesis of mono-unsaturated fatty acids, was downregulated in MIRKO muscle (Table 1). This downregulation would be expected to decrease palmitoleate (16:1) and oleate (18:1) synthesis, which is a change that could contribute to changes in membrane fluidity (a feature of diabetes and insulin resistance) (Vessby, B., (2000) Br. J. Nutr. 83 Suppl. 1, S91-S96).

### Transcription- and Translation-Related Genes

Histone acetyl transferase (HAT) type B was decreased by 41%. HAT activity, especially that associated with CBP/p300, is crucial in differentiation of skeletal muscle (Polesskaya et al., (2001) EMBO J. 20, 6816-6825). Downregulator of transcription DR-1 was upregulated by 110% in MIRKO muscle. DR-1 is a phosphoprotein that interacts with the TATA box-binding protein (TBP), and represses both basal and activated levels of transcription (Inostroza et al., (1992) Cell 70, 477-489; White et al., (1994) Science 266, 448-450). DR-1 was further upregulated in diabetes (see below).

### Other Genes

Expression of NSF (N-ethylmaleimide-sensitive fusion) protein and VAMP-2 (vesicle associated membrane protein 2), which have been implicated in Glut4 translocation (St-Denis and Cushman, (1998) J. Basic Clin. Physiol. Pharmacol. 9, 153-165; Bryant et al., (2002) Nat. Rev. Mol. Cell. Biol. 3, 267-277), was decreased significantly in MIRKO muscle. Interestingly, insulin-degrading enzyme (IDE) an extracellular thiol metalloprotease (which is capable of degrading insulin, insulin-like growth factors I and II, transforming growth factor type α, and β-amyloid (Hamel et al., (1997) Biochim. Biophys. Acta 1338, 207-214, Qiu et al., (1998) J. Biol. Chem. 273, 32730-32738)) is downregulated in the MIRKO muscle. IDE has been associated also with the diabetic phenotype in GK rats (Fakhrai-Rad et al., (2000) Hum. Mol. Genet. 9, 2149-2158), and a deletion of this gene in mice resulted in hyperinsulinemia, glucose intolerance, and increased cerebral accumulation of endogenous 13-amyloid, which is a hallmark of Alzheimer's disease (Farris et al., (2003) Proc. Natl. Acad. Sci. USA 100, 4162-4167).

### Comparison of Lox-STZ and MIRKO and MIRKO-STZ

Data from Lox control mice, MIRKO mice, and MIRKO mice made diabetic with STZ were compared to determine the direct effects of insulin versus the effects of the diabetic state on gene expression. In contrast with the modest number of changes (n=130) in gene expression in the MIRKO mouse, the induction of diabetes by STZ led to many changes in gene expression in both the Lox control vs. Lox-STZ (n=512) and MRKO vs. MIRKO-STZ (n=487) comparisons (FIG. 3B). By comparing the genes that were changed significantly in muscle of the diabetic groups (Lox-STZ and MIRKO-STZ) but not changed significantly in muscle of MIRKO mice, genes were identified that were regulated by the diabetic state (e.g., regulated by altered metabolism, hormones, or glycation) as opposed to the loss of insulin-receptor signaling.

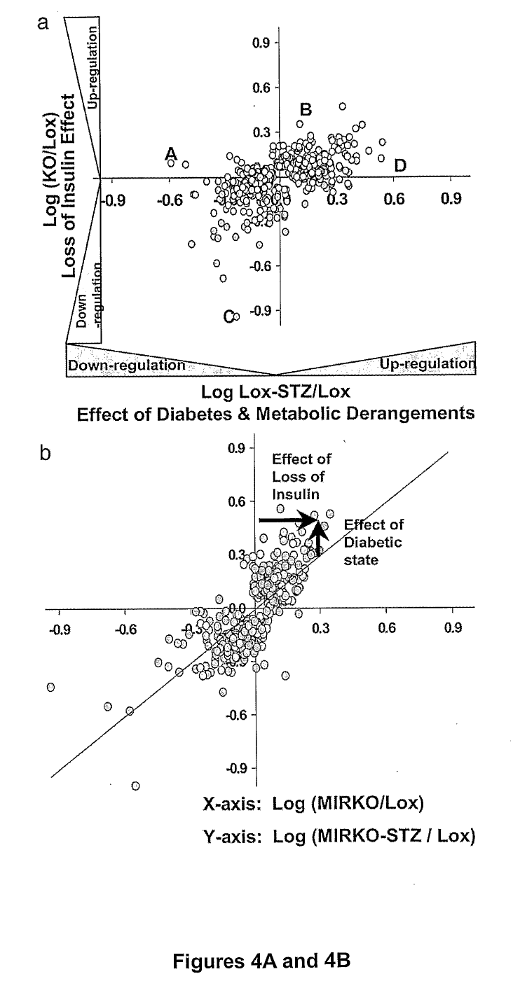

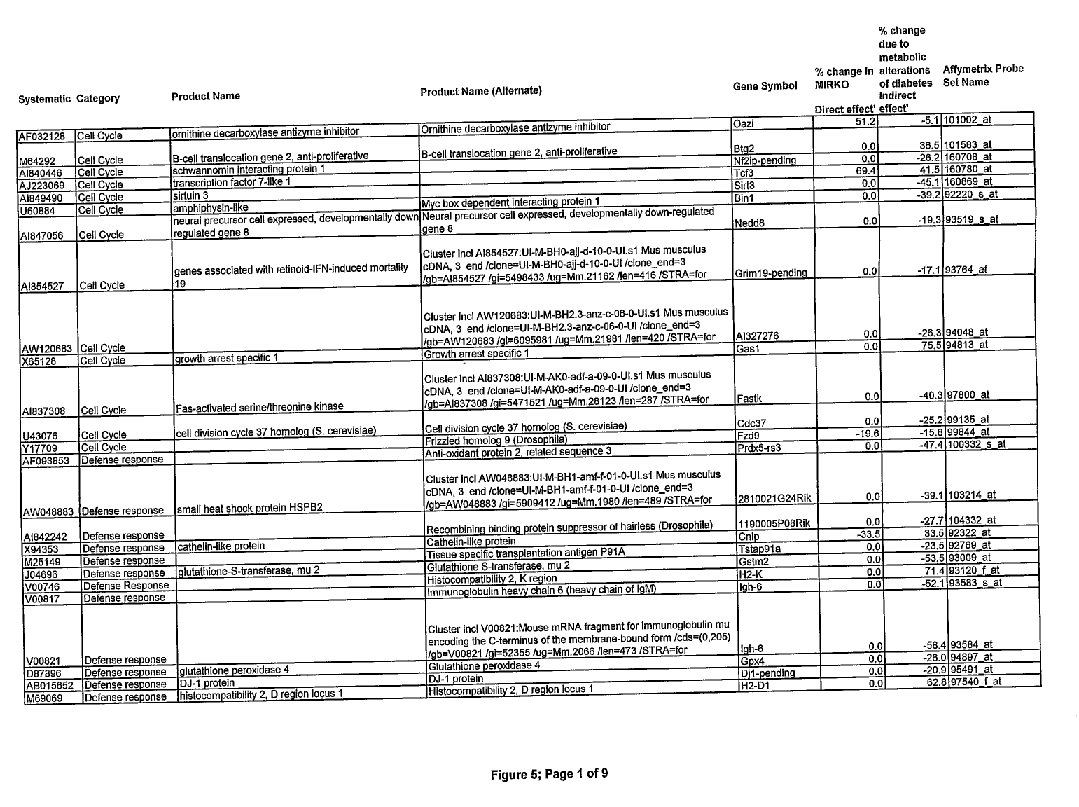

Genes that were changed significantly in MIRKO versus Lox-STZ mice and the MIRKO versus MIRKO-STZ mice are shown in FIG. 4. In FIG. 4A, the ratio of expression for (MIRKO/Lox) (plotted on the ordinate) represents the effect of an isolated loss of insulin signaling on gene expression, whereas the ratio of expression for (Lox-STZ/Lox) (plotted on the abscissa) represents the combined effect of a loss of insulin signaling due to insulin deficiency and the diabetic state with all of its metabolic consequences. This analysis reveals both the concordance and discordance of the effects of diabetes and the effects of an isolated loss of insulin action.

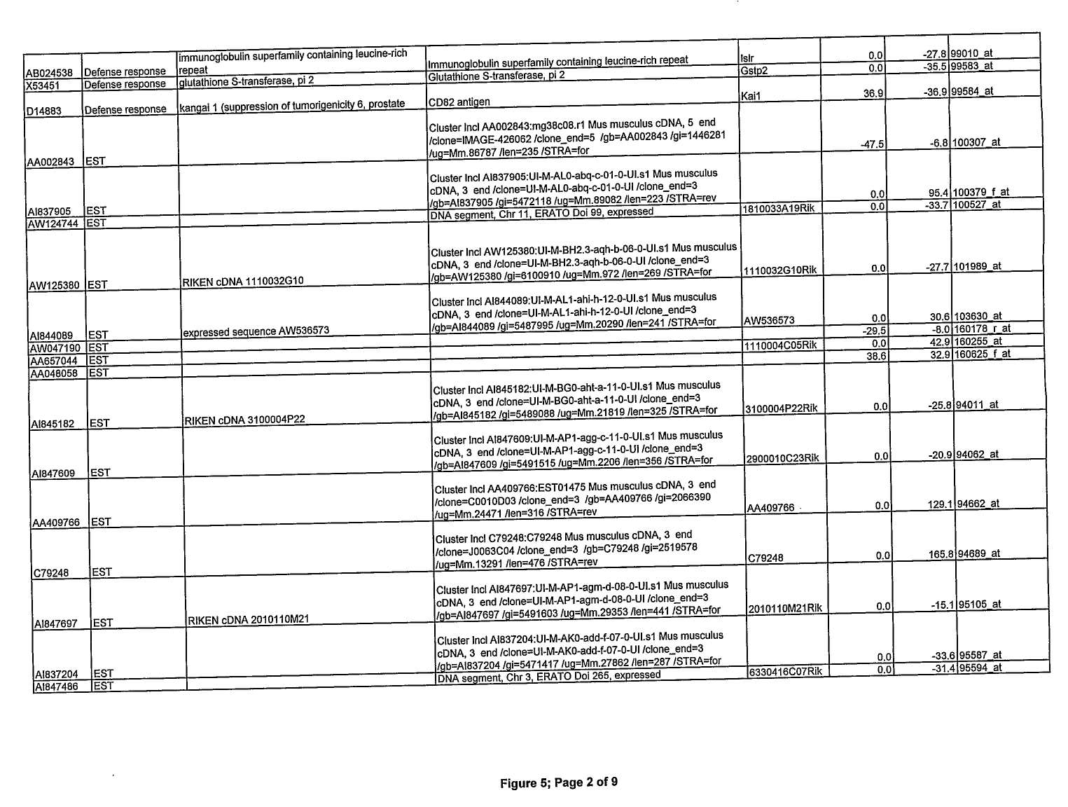

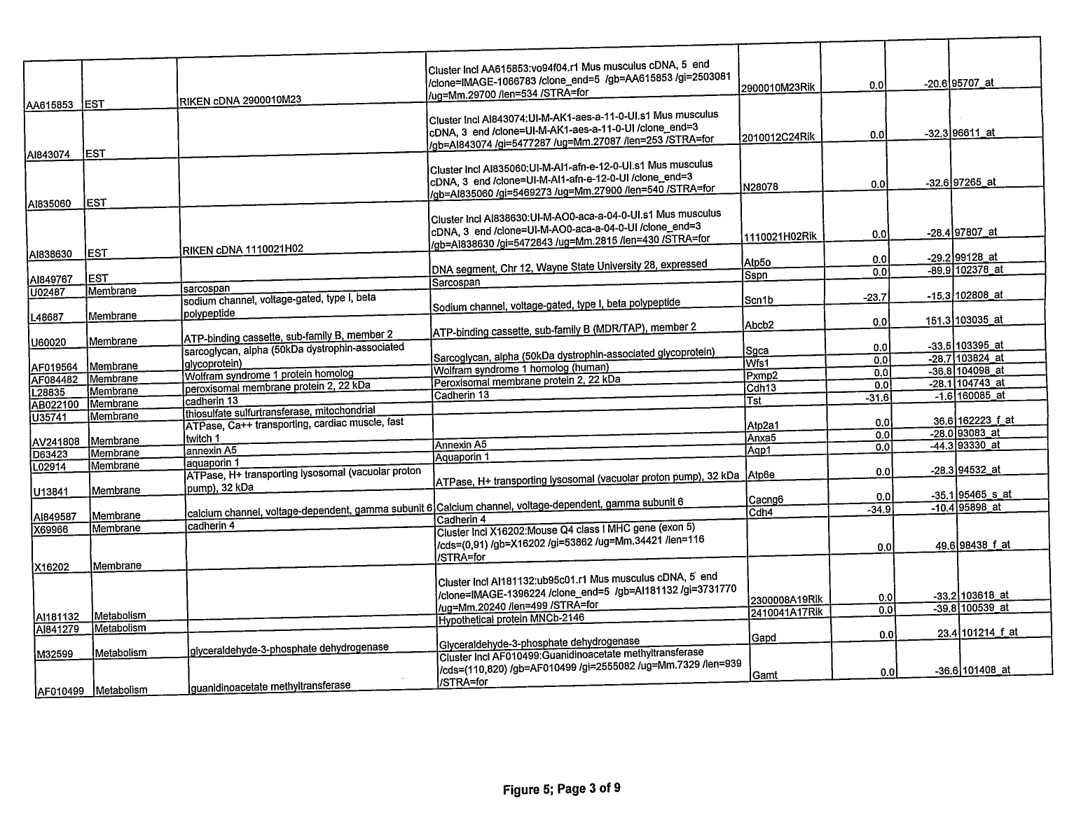

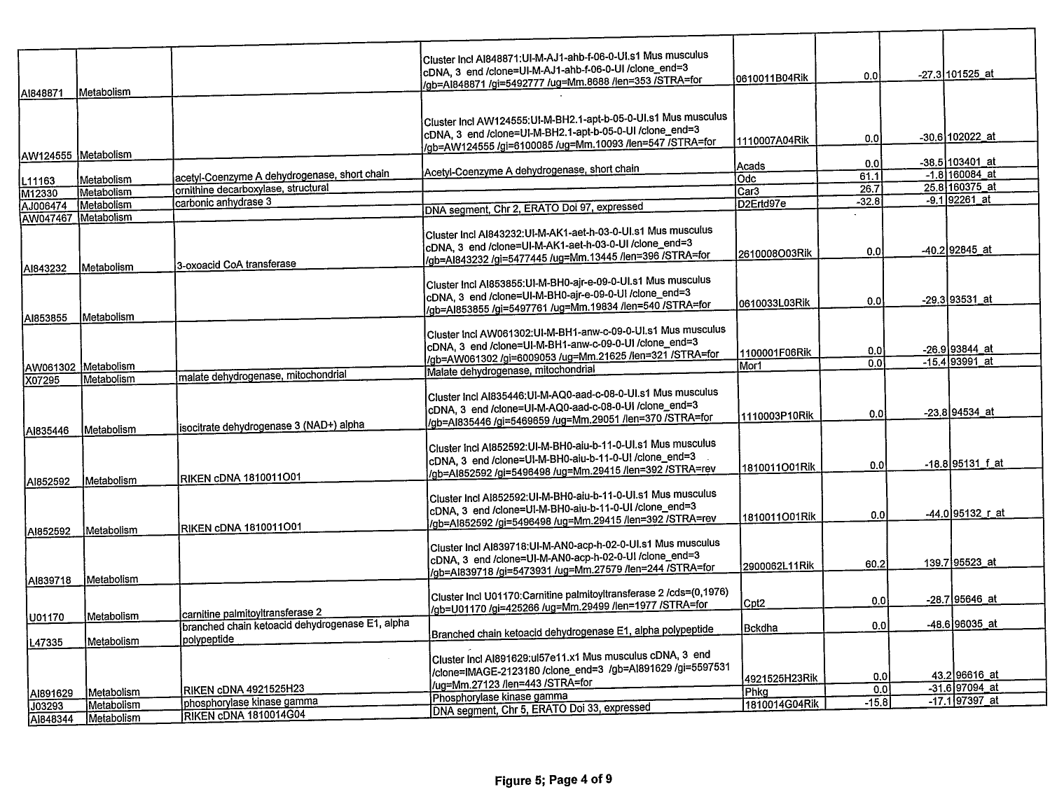

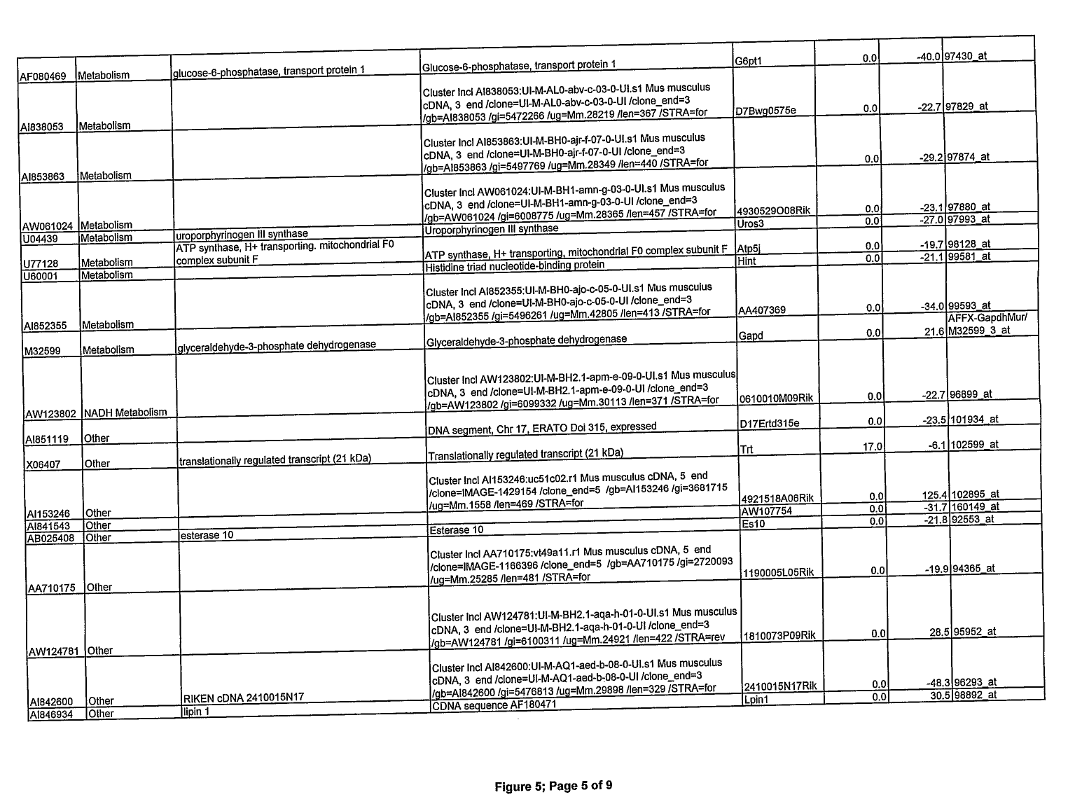

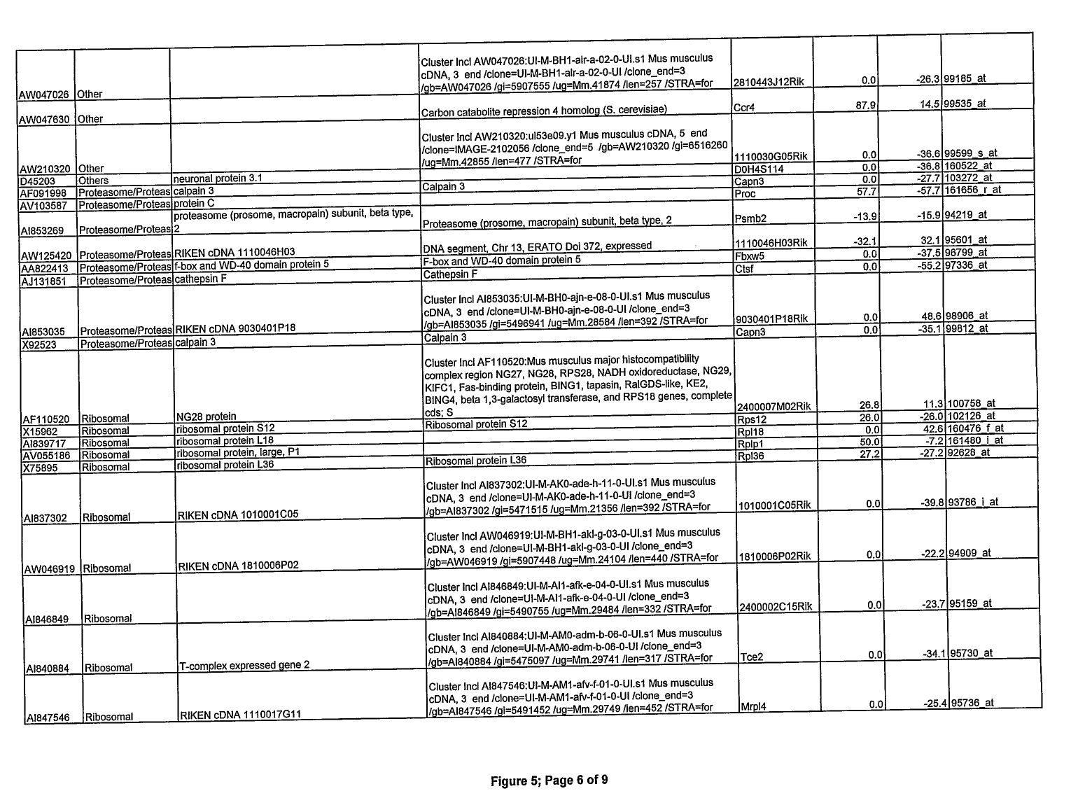

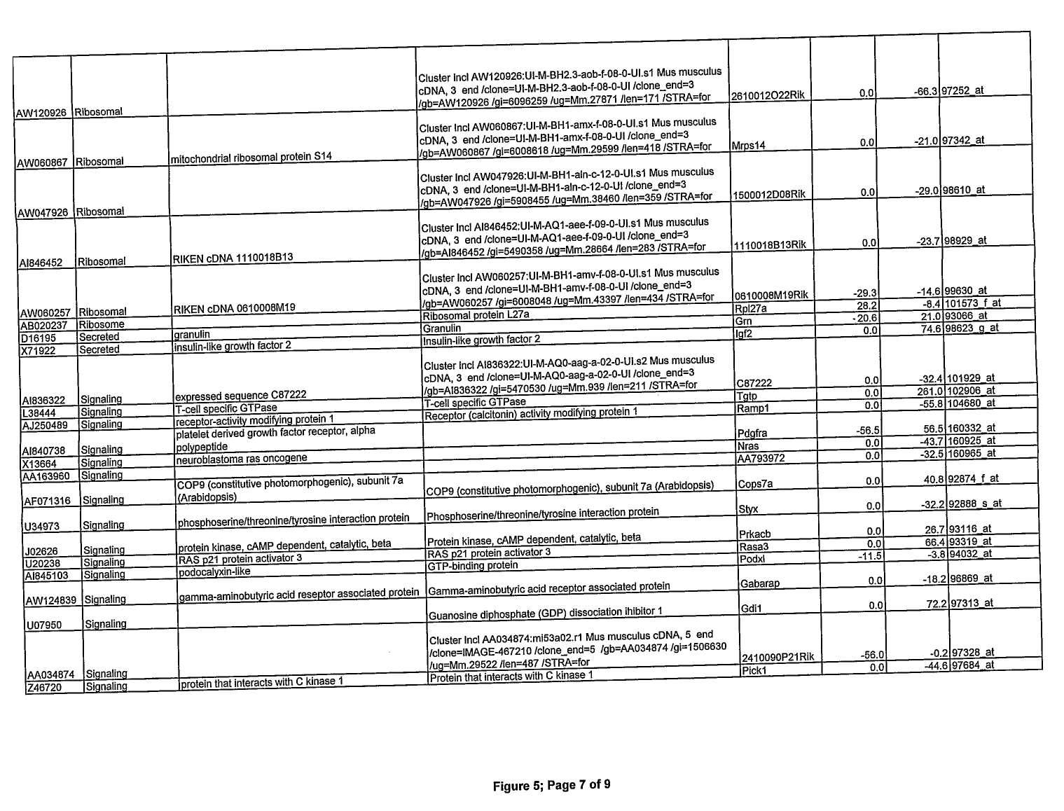

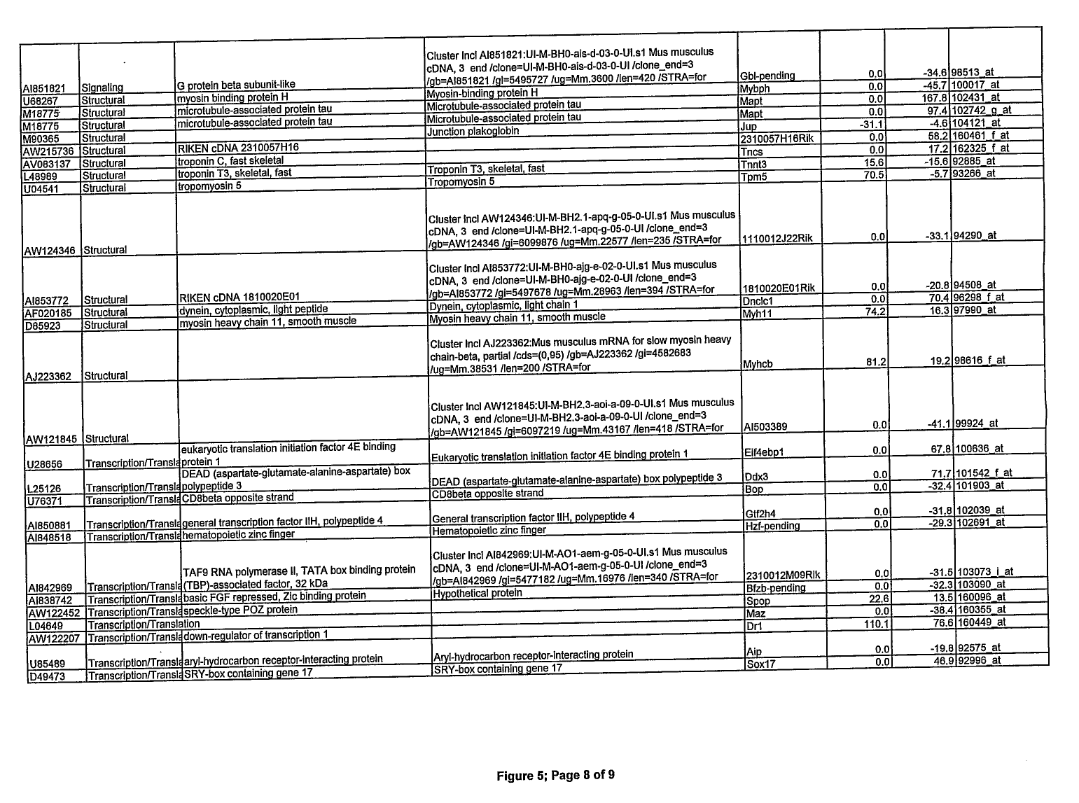

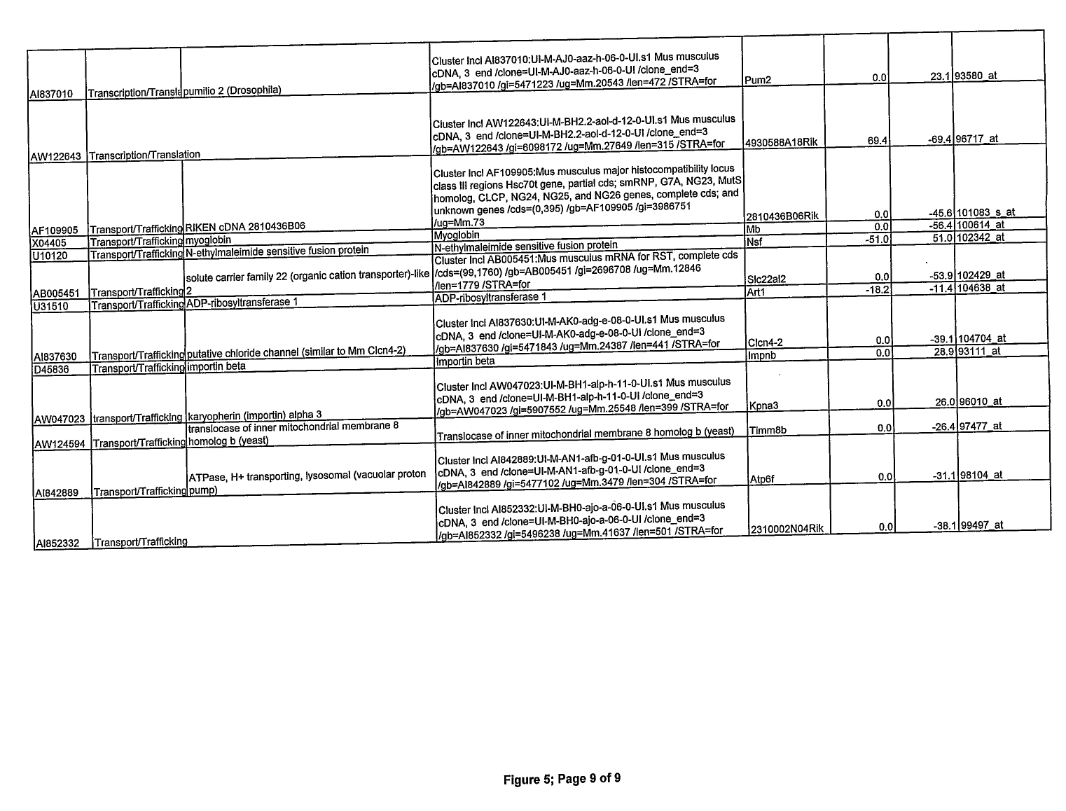

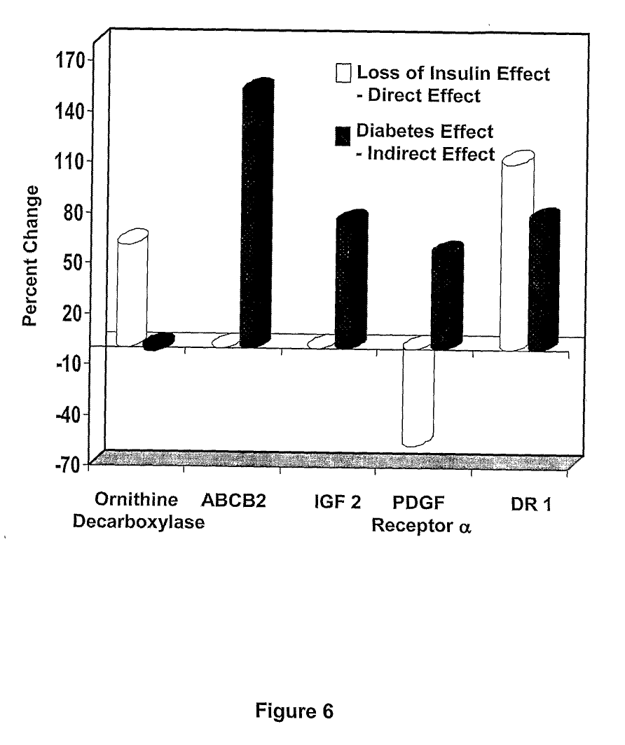

A similar comparison of MIRKO and MIRKO-STZ versus Lox controls is shown in FIG. 4B. In this case, by calculating the extent to which the points in the graph deviate from the line of identity (as shown by arrows for a representative gene in FIG. 4B, one can dissect out the respective contributions of insulin signaling and diabetes on gene expression (arrows in FIG. 4B). This analysis is presented in the table of FIG. 5, and some examples are shown in FIG. 6. Expression of ornithine decarboxylase was upregulated in the MIRKO mouse but was not changed further by induction of diabetes. However, the ATP-binding cassette B2 gene and insulin-like growth factor II were both upregulated by diabetes but not changed in the MIRKO mouse. Platelet-derived growth factor receptor a was downregulated in the MIRKO mouse, but induction of diabetes had a nearly equal effect to upregulate the gene. Thus in the MIRKO-STZ mouse, levels of this mRNA were essentially normal. Also, the loss of insulin action (MERKO muscle) and diabetes both upregulated DR1, such that the levels in the MIRKO-STZ mouse were even higher than in either STZ or MIRKO animals.

### Analysis of Diabetes-Regulated Genes

From the above data set, 205 (118 downregulated and 87 upregulated) genes were identified that were differentially expressed in both diabetes models but not regulated in the MIRKO mouse (FIG. 3B). By comparing the changes induced by diabetes in the MIRKO mice (MIRKO-STZ) with those in Lox-STZ mice and then studying which are correctable by insulin treatment, two striking patterns of insulin-regulated versus diabetes-regulated gene expression could be identified.

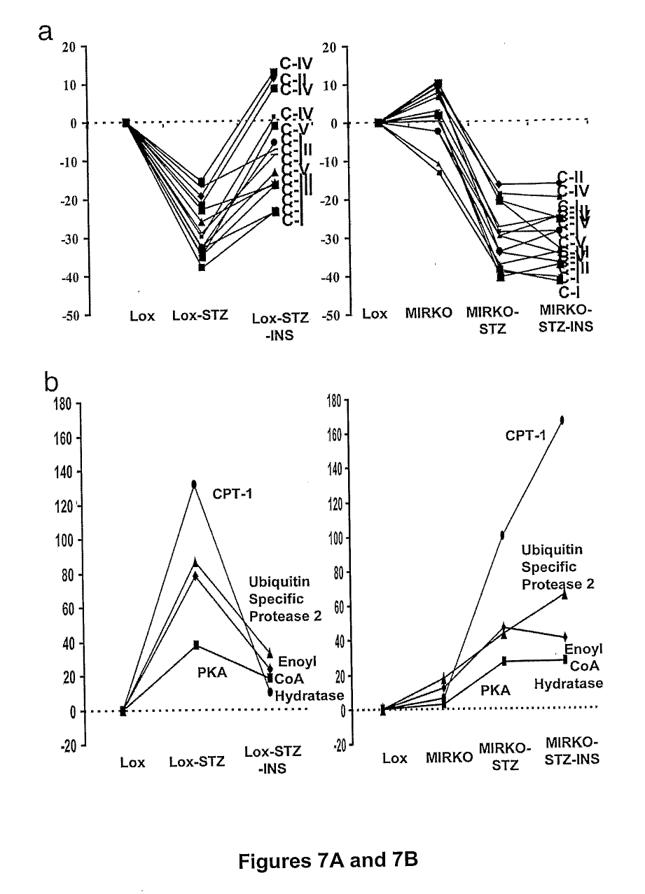

The first pattern is exemplified by genes that (i) were normal in the MIRKO mouse but were downregulated in diabetes and (ii) responded to insulin treatment only in the Lox-STZ mice and not in the MIRKO-STZ (FIG. 7A and Table 3). Of these, 19 genes were metabolism-related, including 12 transcripts encoding the electron-transport chain. Although the decreases in expression were often modest (15-34%), they were highly reproducible, statistically significant, and coordinate in direction. This study reveals a mechanism for this coordinated transcriptional regulation (FIG. 7 and see below) because all of these genes were downregulated significantly in the diabetic Lox-STZ and MIRKO-STZ mice, and none were significantly changed in the MIRKO group (i.e., these are diabetes-regulated, not insulin-regulated, genes, but insulin action was required for return of the diabetic defect toward normal). A list of genes that were regulated in a similar way is presented in Tables 3 and 4.

TABLE 3
Genes significantly downregulated in diabetes with intact (third column)
or without (fourth column)insulin-receptor-mediated signaling.

| | | | |
|---|---|---|---|
| GenBank | | | |
| accession | | Fold change in | Fold change in |
| no. | Gene_protein name | Lox-STZ_Lox | MIRKO-STZ_Lox |

Metabolism-related

| | | | |
|---|---|---|---|
| AI843232 | 3-Oxoacid CoA transferase | 0.55 | 0.60 |
| AW047743 | Isovaleryl coenzyme A dehydrogenase | 0.63 | 0.70 |
| AI853855 | Complex I | 0.66 | 0.70 |
| AF010499 | Guanidinoacetate methyltransferase | 0.66 | 0.63 |
| | (creatine synthesis) | | |
| AI181132 | Creatine kinase precursor, mitochondrial | 0.67 | 0.67 |
| AF080469 | Glucose-6-phosphatase, transport | 0.68 | 0.60 |
| | protein 1 | | |
| AI848871 | Complex I | 0.69 | 0.73 |
| U13841 | Complex V | 0.69 | 0.72 |
| AW123802 | Complex I | 0.71 | 0.77 |
| U15541 | Complex IV | 0.71 | 0.76 |
| AI849803 | Complex I | 0.74 | 0.77 |
| AI849767 | Complex V | 0.74 | 0.71 |
| AF029843 | Phosphoglycerate mutase muscle- | 0.77 | 0.83 |
| | specific subunit | | |
| AI853523 | Complex III | 0.78 | 0.70 |
| AF037371 | Complex IV | 0.79 | 0.80 |
| X53157 | Complex IV | 0.82 | 0.82 |
| AW061302 | Complex III | 0.84 | 0.73 |
| U77128 | Complex V | 0.88 | 0.80 |
| AI852862 | Fumarate hydratase 1 | 0.90 | 0.90 |

Signaling-related

| | | | |
|---|---|---|---|
| AI836322 | Similar to RhoGDI-1 | 0.66 | 0.68 |

Transcription/translation-related

| | | | |
|---|---|---|---|
| M98036 | Eukaryotic translation initiation factor 2B | 0.75 | 0.77 |
| AI854467 | SD23 homolog | 0.77 | 0.79 |

A similar but inverse profile of gene expression (i.e., upregulated in diabetes (Lox-STZ and MIRKO-STZ) but with no significant change in MIRKO, and responsive to insulin only in the Lox-STZ but not in the MIRKO-STZ) was observed for 33 genes (FIG. 7B and Table 4). This pattern of transcriptional regulation was operative for many genes involved in fatty-acid metabolism, including carnitine palmitoyl transferase 1, 43-2 enoyl CoA isomerase, acetyl CoA synthetase 2, and monoglyceride lipase. The transcript of cAMP-specific protein kinase β catalytic subunit (which is upregulated in Lox-STZ and MIRKO-STZ) has multiple metabolic actions, including in glycogen metabolism in which it opposes insulin action. Interestingly, decreased activity of this enzyme is associated with increased longevity in yeast (Lin et al., (2002) Nature 418, 344-348).

TABLE 4
Genes significantly upregulated in diabetes with intact (third column)
or without (fourth column) insulin-receptor-mediated signaling.

| | | | |
|---|---|---|---|
| GenBank | | | |
| accession | | Fold change in | Fold change in |
| no. | Gene_protein name | Lox-STZ_Lox | MIRKO-STZ_Lox |

Metabolism-related

| | | | |
|---|---|---|---|
| AF017175 | Carnitine palmitoyltransferase 1, liver | 2.31 | 2.00 |
| AI840013 | Peroxisomal delta3, delta2-enoyl- | 1.78 | 1.64 |
| | coenzyme A isomerase | | |
| AW125884 | Acetyl-coenzyme A synthetase 2 | 1.49 | 1.50 |
| AI846600 | Monoglyceride lipase | 1.24 | 1.27 |

Signaling-related

| | | | |
|---|---|---|---|
| AI836322 | Protein tyrosine phosphatase, | 1.76 | 1.49 |
| | nonreceptor type 1 | | |
| AW049031 | Immediate-early response, erythropoietin 1 | 1.73 | 1.72 |
| M19381 | Calmodulin | 1.42 | 1.26 |
| J02626 | Similar to protein kinase, cAMP- | 1.37 | 1.27 |
| | dependent, catalytic, β | | |
| U22324 | Fibroblast growth-factor receptor 1 | 1.28 | 1.35 |

Transcription/translation-related

| | | | |
|---|---|---|---|
| AF038939 | Paternally expressed gene 3 | 1.89 | 1.88 |
| AA960603 | Butyrate response factor 2 | 1.52 | 1.54 |
| AI846060 | Zinc finger RNA binding protein | 1.47 | 1.47 |
| U00431 | High-mobility group protein 1 | 1.43 | 1.30 |
| AI835685 | Splicing factor pRP 8 | 1.4 | 1.30 |
| X98511 | Similar to splicing factor, arginine/serine- | 1.39 | 1.43 |
| | rich 2 (SC-35) | | |

Transport/trafficking-related

| | | | |
|---|---|---|---|
| AI839718 | Microsomal signal peptidase 23 kDa | 2.12 | 3.00 |
| AI843574 | Homolog to signal recognition particle α | 1.44 | 1.17 |
| | subunit (docking protein α) | | |
| AI835359 | Translocon-associated protein α (TRAP- | 1.39 | 1.33 |
| | α), signal sequence receptor α | | |

### Regulation of Transcription and Translation by DR-1, HAT Type B, and Sirtuin3

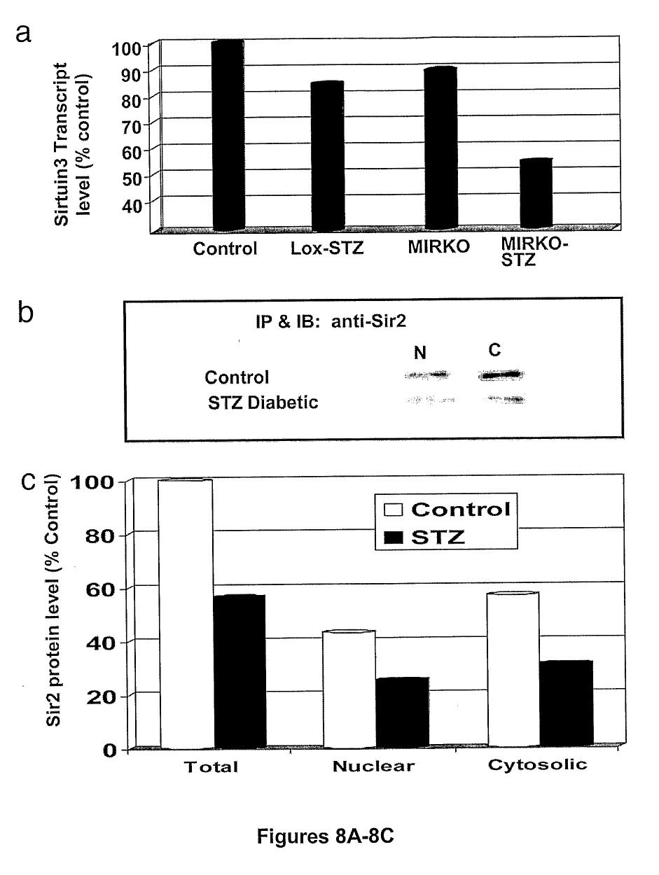

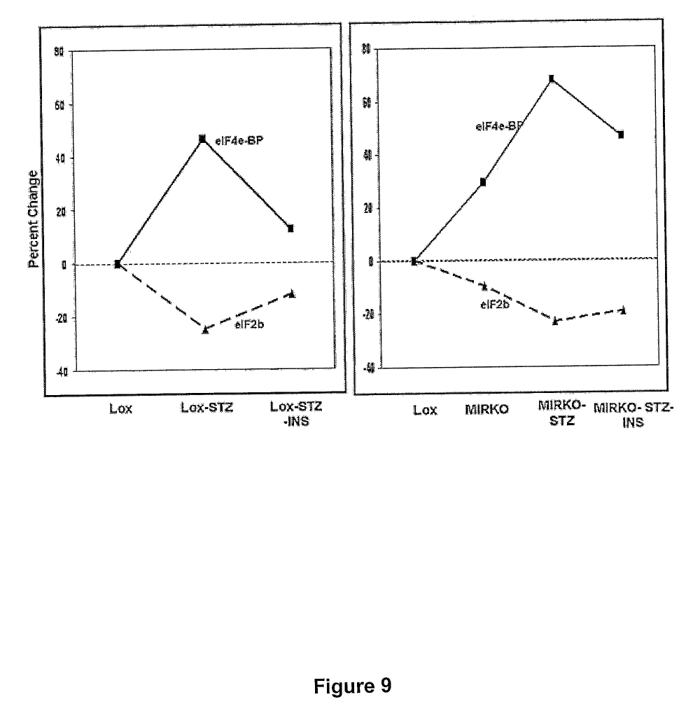

Several components of the general transcription and translation machinery were altered in diabetes. In addition to the upregulation of DR-1 and downregulation of HAT type B that was described above, sirtuin3 (a mouse homolog of the yeast silent mating type information regulator 2 (Sir2)) was downregulated significantly in the MIRKO-STZ. It also decreased in the Lox-STZ, although this change did not achieve statistical significance. Sir2 is a family of type III histone deacetylases that are involved in NAD-dependent transcriptional repression. Western blotting confirmed that protein levels of Sir2 homologues in the nuclear/mitochondrial and cytosolic extracts from skeletal muscle of STZ diabetic mice were decreased by 40-45% (FIG. 8). mRNA for eukaryotic translation initiation factor (eIF) 2b 8 subunit was also decreased in the two diabetic states (Lox-STZ and MIRKO-STZ), whereas that the translation inhibitor eIF4e-binding protein (eIF4e-bp) was increased in MIRKO muscle and increased even more when diabetes was superimposed on this model (FIG. 9). The activity of eIF4e-bp has been shown to be regulated by insulin through a phosphorylation cascade (Gingras et al., (1998) Genes Dev. 12, 502-513) and is decreased in diabetes (Kostyak et al., (2001) J. Appl. Physiol. 91, 79-84). In addition, eIF4e-bp has been linked to insulin resistance because deletion of this gene results in increased insulin sensitivity (Tsukiyama-Kohara et al., (2001) Nat. Med. 7, 1128-1132).

### Example 2

### Sirtuin2

Sir2 is a Class III NAD-dependent histone deacetylase that mediates transcriptional silencing at mating-type loci, telomeres, and ribosomal gene clusters. Sir2 homologues have been identified in yeast, bacteria, Caenorhabditis elegans, Drosophila, and mammals; Sir2 has a critical role in the determination of lifespan in yeast and Caenorhabditis elegans. Mammalian sirtuin2 protein is predominately located in cytoplasm, has been implicated in cell cycle control and cytoskeleton organization, and can interact with other transcription factors to regulate gene expression. The human sirtuin2 gene is on chromosome 7. Sirtuin2 deacetylates monoacetylated histone H3 and H4 peptides and tubulin substrates. Expression is downregulated in gliomas.

Sirtuin2 is phosphorylated late in G(2), during M, and into the period of cytokinesis. CDCl₄B may provoke exit from mitosis coincident with the loss of sirtuin2 via ubiquitination and subsequent degradation by the 26S proteasome.

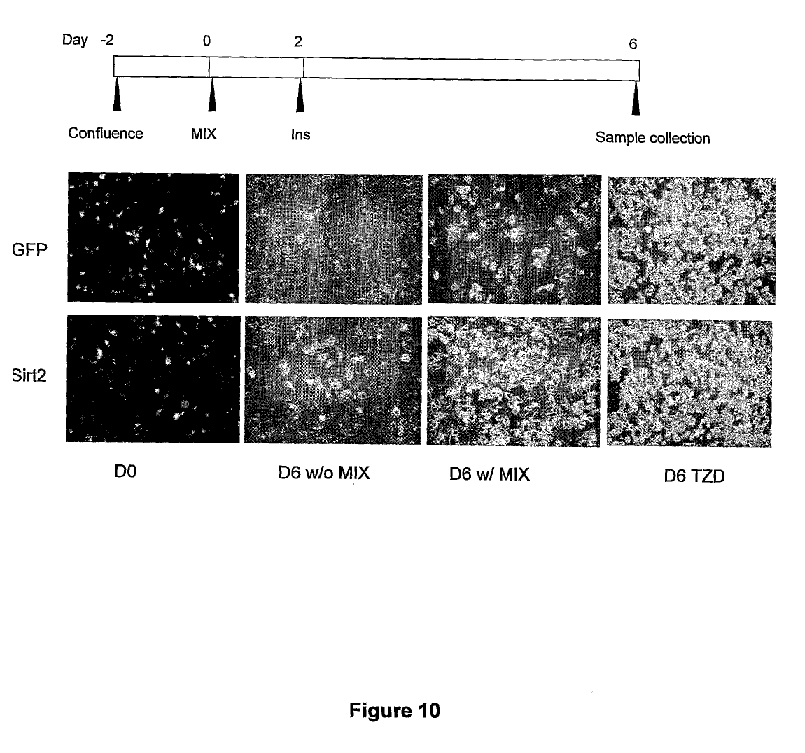

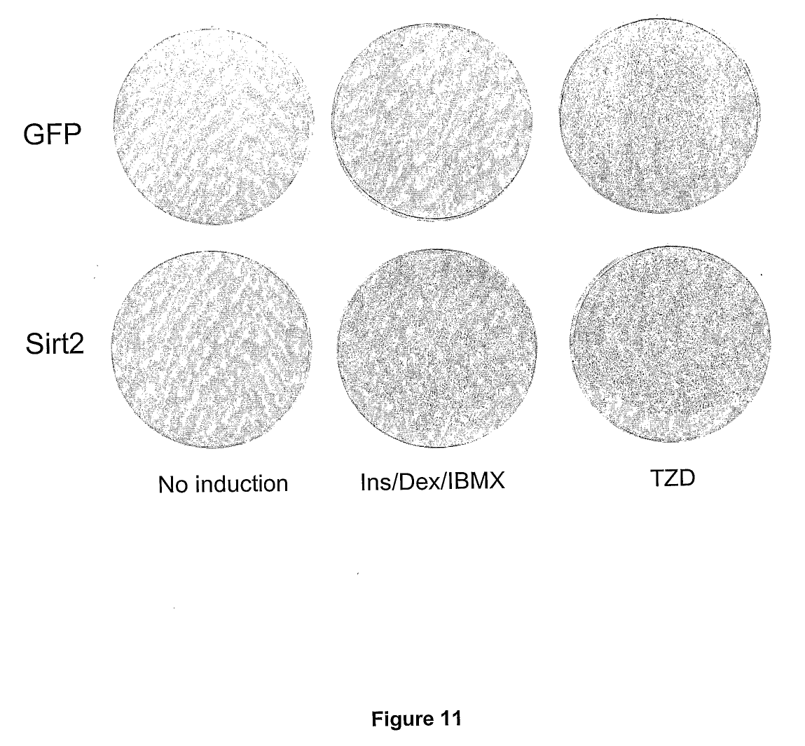

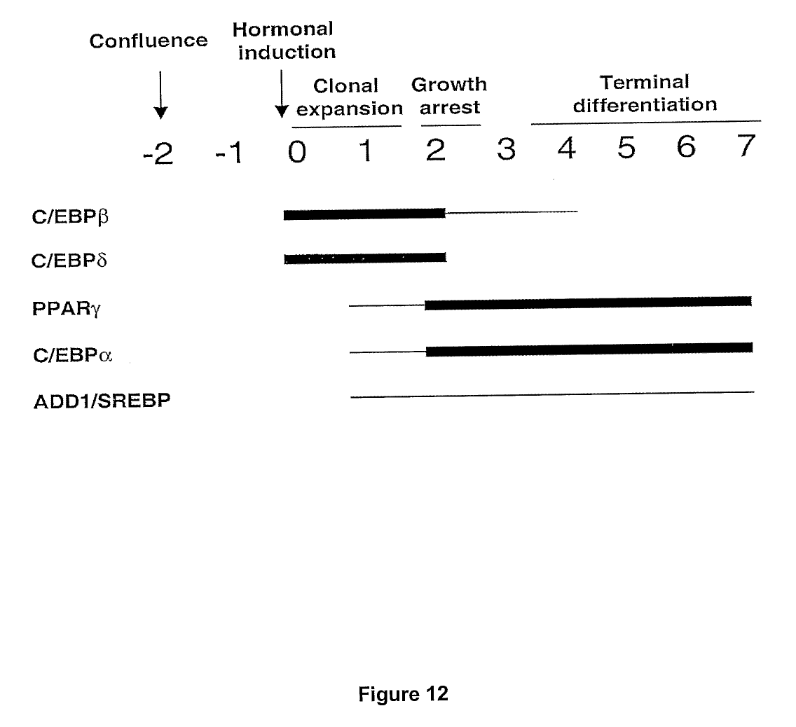

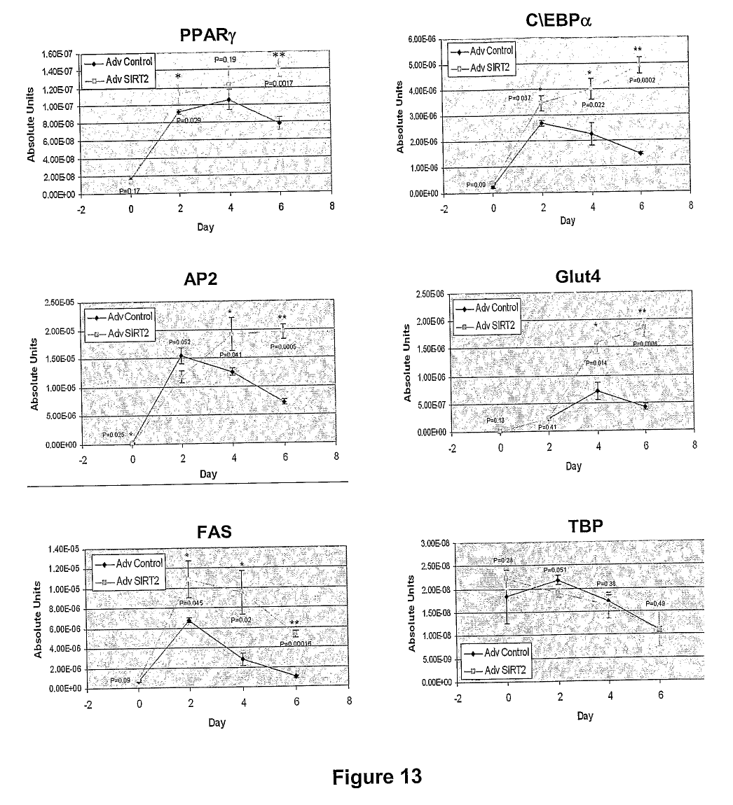

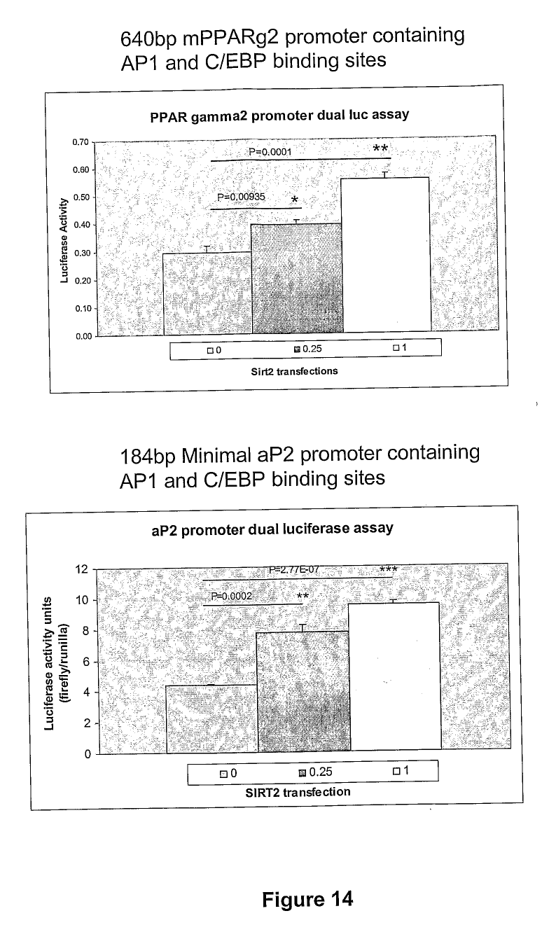

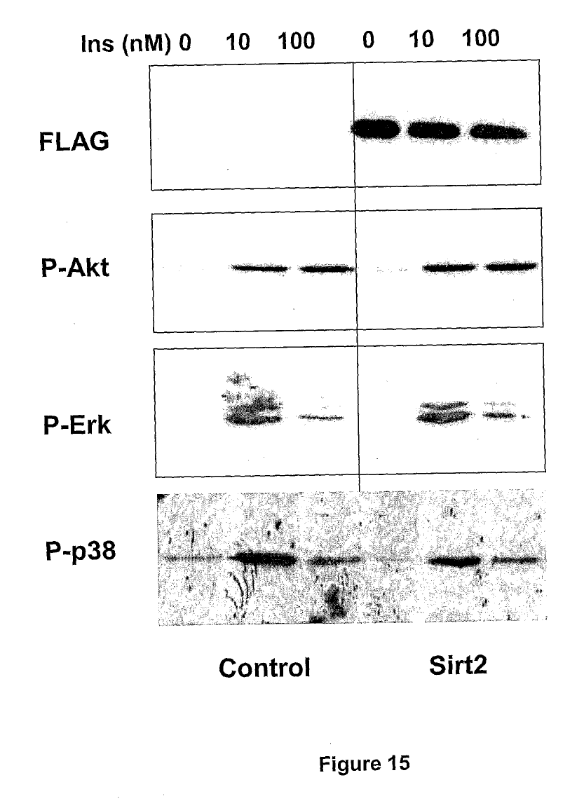

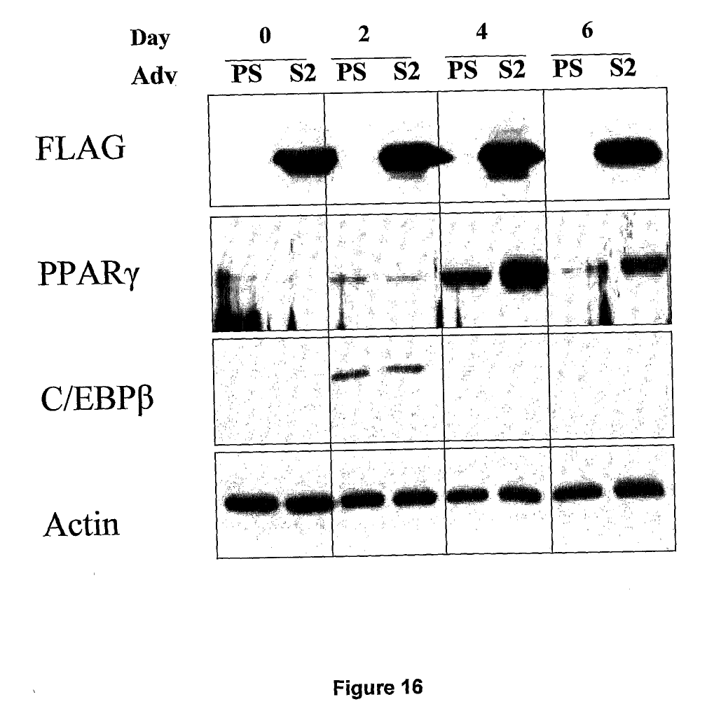

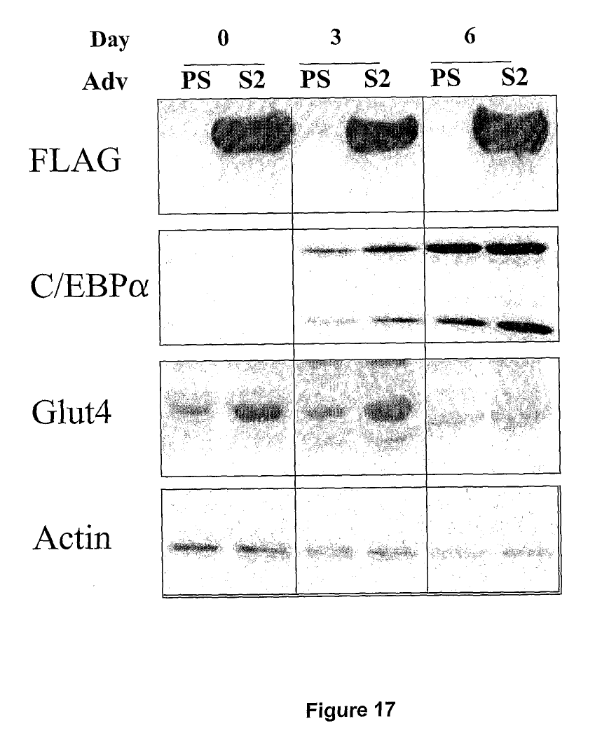

We have analyzed the role of mouse sirtuin2 (SEQ ID NO:4) in adipocyte differentiation and determined its mechanism of action via interaction with transcriptional control elements such as PPARγ and C/EBPα. By using adenoviral gene transfer, recombinant full-length mouse sirtuin2 was introduced into CAR cells (3T3 L1 pre-adipocytes that have adenoviral receptor over-expressed to enhance the infection) and C3H10 cells. These cells were compared with cells infected with control virus containing GFP. The mouse sirtuin2 overexpressing cells displayed significantly higher differentiation ability as compared to the GFP-expressing cells (FIGS. 10 and 11). Temporal expression of major transcription factors during adipogenesis are shown (FIG. 12). The mRNA and protein expression levels of different adipocyte differentiation markers such as fatty acid synthase (FAS), Glut4, and aP2 were significantly promoted by mouse sirtuin2 overexpression (FIG. 13). Promoter activity assays (FIG. 14) showed that mouse sirtuin2 had a significant effect on both PPARγ and aP2 promoters (2-3 fold), indicating that mouse sirtuin2 interacts with these promoters directly or indirectly and regulates their downstream gene expression, and thus promotes adipogenesis in 3T3 L1 preadipocytes. Sirtuin2 had no effect on insulin signaling in terms of Ras-MAPK and P13 Kinase-Akt pathways (FIG. 15), but appeared to regulate insulin sensitivity by modulating downstream gene expression such as PPARγ and C/EBPα (FIGS. 16 and 17). These results indicate that sirtuin2 is important for adipocyte differentiation and blocking the activity or expression of sirtuin2 is therefore useful for the treatment of obesity.

### Reduction of Sirtuin2 Expression by RNAi Decreases Adipogenesis in C3H10 Cells

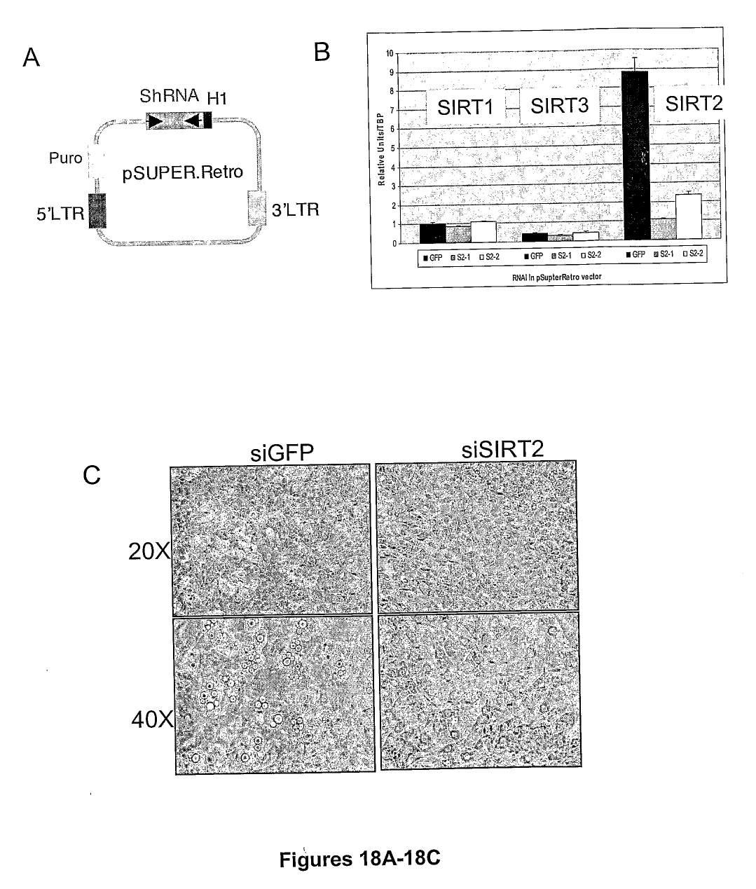

Expression of Sirtuin2 in C3H10 cells was reduced by transfection with a pSuper.Retro vector encoding a small, inhibitory RNA (siRNA) that binds to sirtuin2, as shown in FIG. 18A. Two RNAi constructs were created, one specific to exon 4 (labeled S2-1) and one specific to exon 9 (labeled S2-2) of mouse sirtuin2. Introduction of these constructs into C3H10 cells resulted in specific reduction of sirtuin2 expression, as compared to either a GFP control construct or as compared to sirtuin1 or sirtuin3 expression (FIG. 18B). Treating C3H10 cells transformed with a siRNA specific for GFP with MIX results in the appears of adipocytes whereas a significant reduction of adipogenesis is observed in cells transformed with an siRNA specific for sirtuin2 (FIG. 18C).

### Reduction of Sirtuin2 Expression by RNAi Increases Expression of Adipogenetic Genes in 3T3L1 Cells

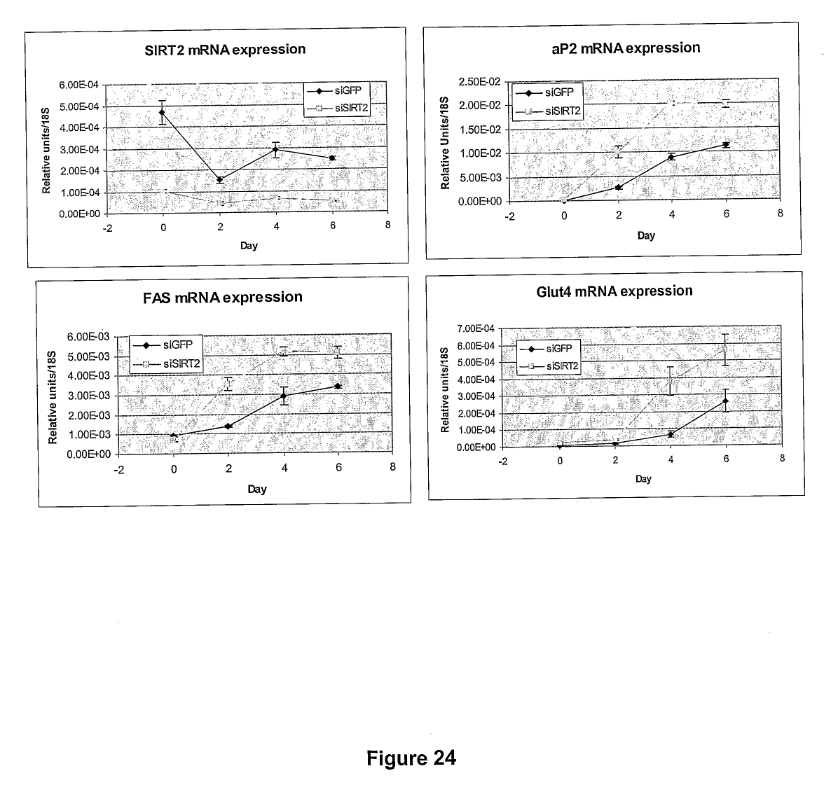

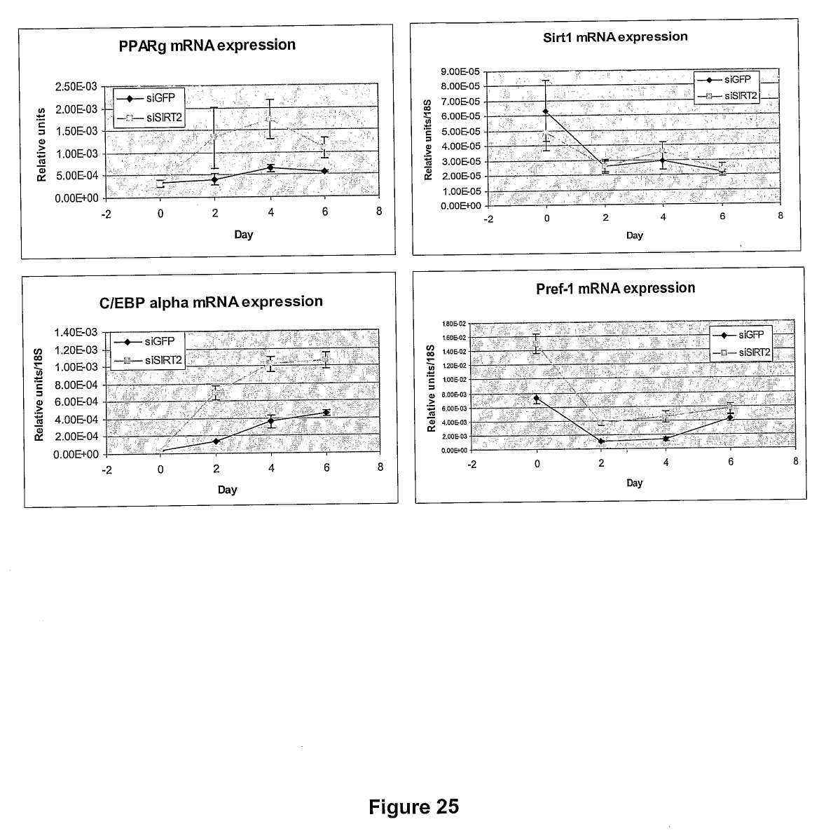

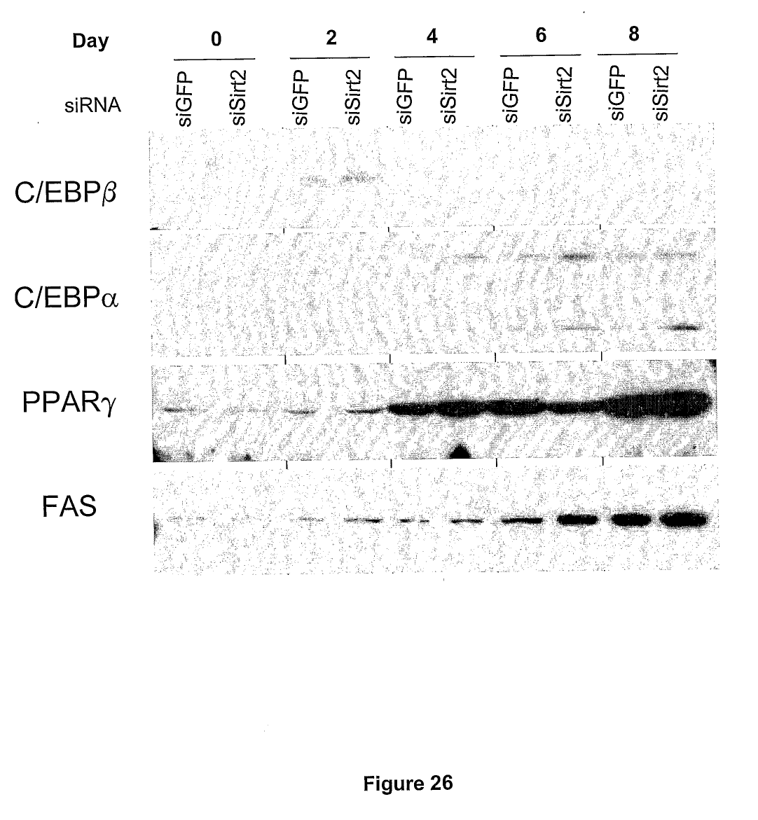

Expression of sirtuin2-targeted siRNA in 3T3L1 cells increases mRNA expression of adipogenetic genes, including aP2, FAS, Glut4, PPARγ, C/EBPα, and Pref-1 (FIGS. 24-25). Protein expression increases in C/EBPβ, C/EBPα, PPARγ, and FAS are also observed (FIG. 26).

### Sirtuin2 Deacetylates and Induces Nuclear Translocation of Foxo1

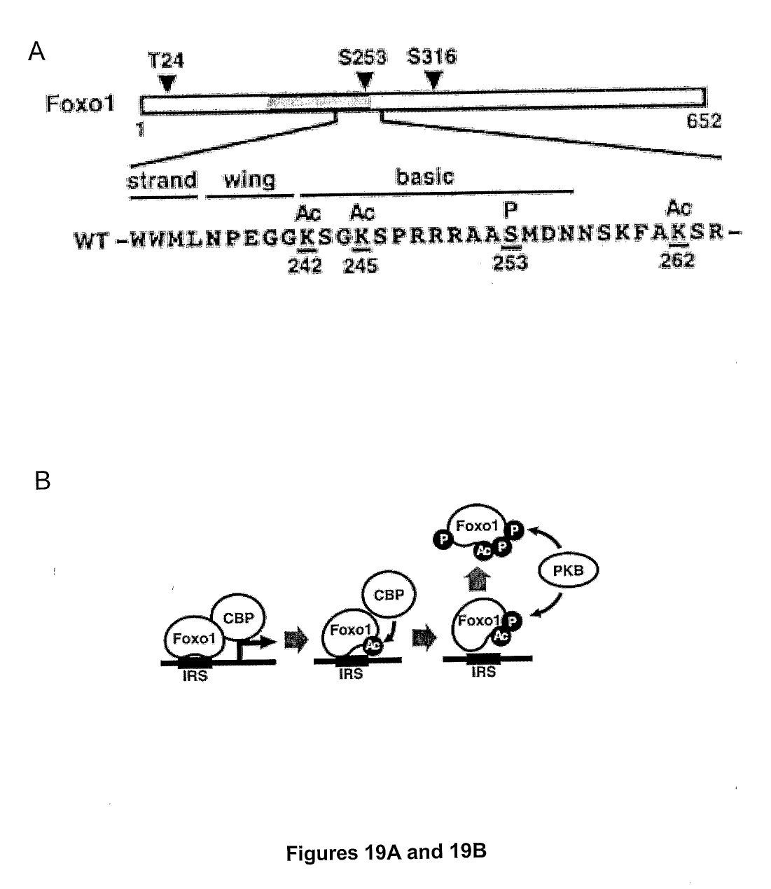

Foxo proteins are transcription factors that contain acetylation and phosphorylation sites that affect their transcription activity (FIG. 19A, which shows Foxo1). Previous work has shown that regulation of Foxo proteins is mediated by CBP, which, in the case of Foxo1, initially induces transcriptional activity but subsequently decreases transcriptional activity by acetylation of Foxo1, as shown in FIG. 19B. Mouse silent information regulator 2 (sirtuin1) has been shown to potentiate Foxo1 transcriptional activity through deacetylation (Daitoku et al., (2004) Proc. Natl. Acad. Sci. USA 101, 10042-10047) and is involved in stress-dependent regulation of Foxo transcription factors. This deacetylation promotes expression of glucogenetic genes. Changes in the acetylation state of Foxo1 are shown to affect its DNA binding, as well as its sensitivity to phosphorylation (Matsuzaki et al., (2005) Proc. Natl. Acad. Sci. USA 102, 11278-11283).

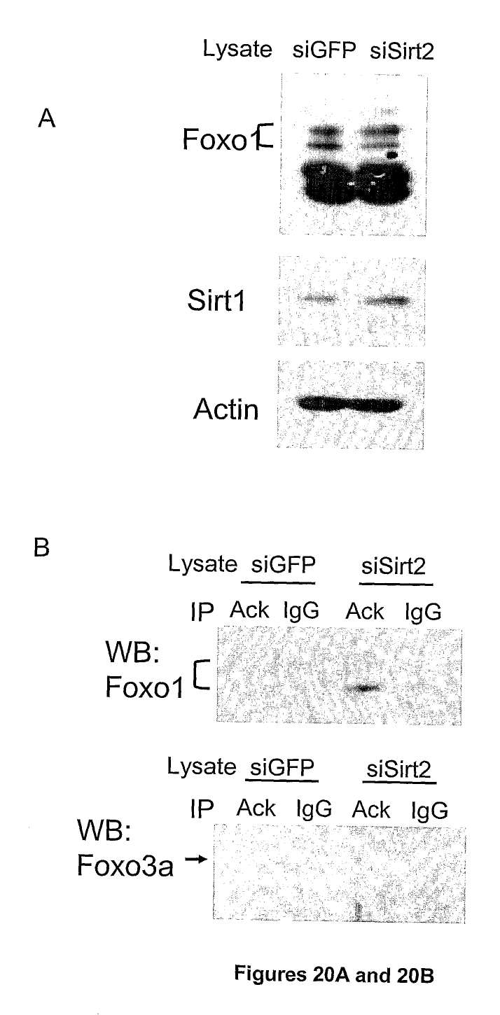

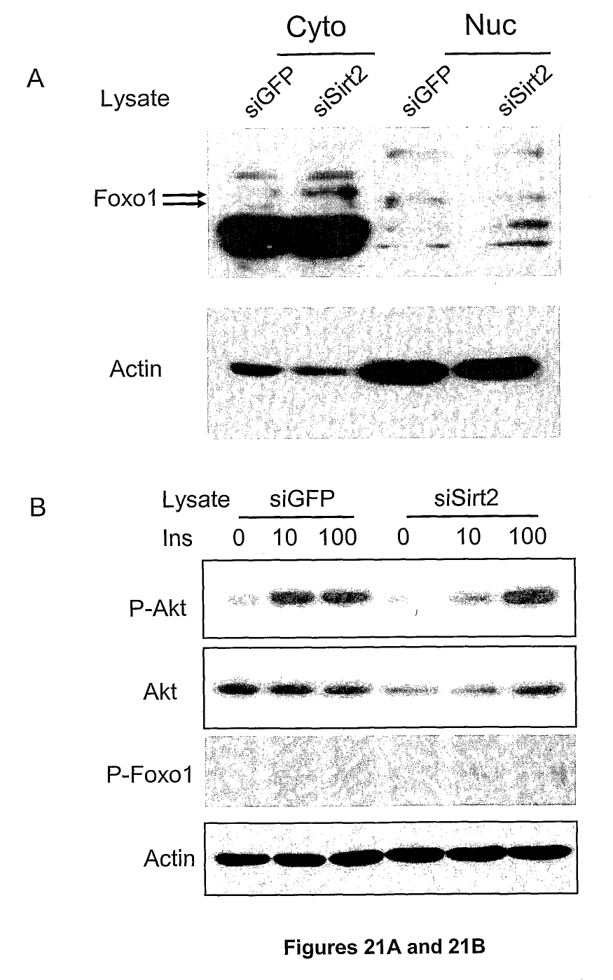

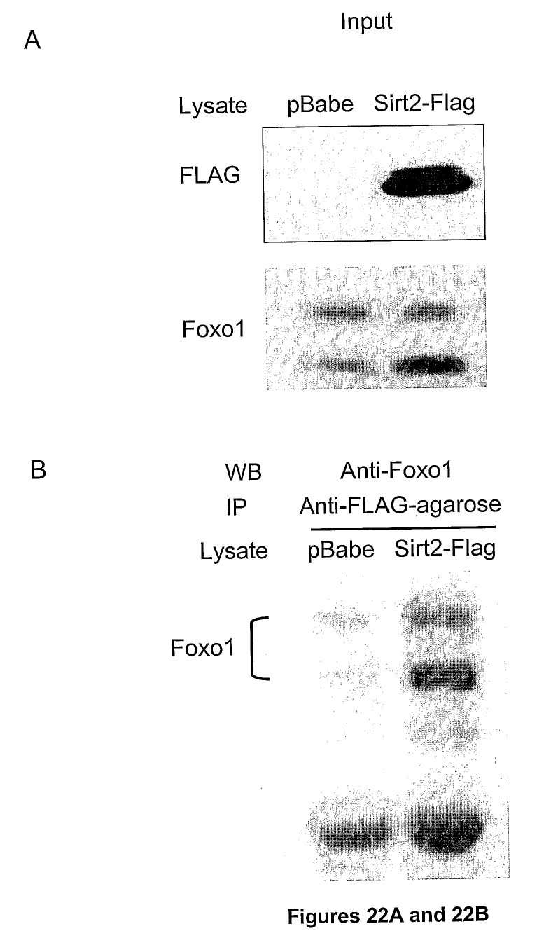

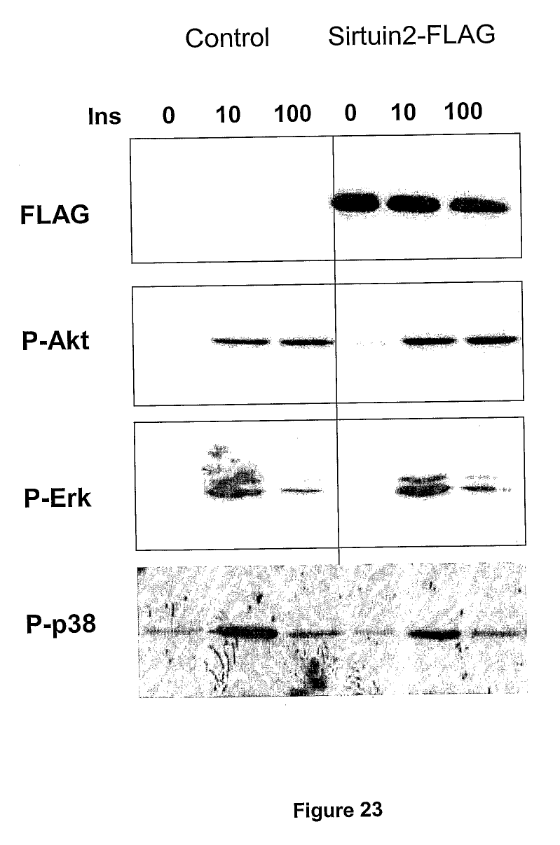

Here, we show that Foxo1 is also deacetylated by sirtuin2 independently of sirtuin1 (FIGS. 20A and 20B), and knockdown of sirtuin2 results in increased acetylation of Foxo1. This increased acetylation, in turn, leads to increased phosphorylation of Foxo1, thereby increasing cytosolic targeting of this protein (FIGS. 21A and 21B). Further, in vitro studies also demonstrate a direct interaction between sirtuin2 and Foxo1 (see FIGS. 22A and 22B). Sirtuin2 activity appears to be independent of insulin signaling, as several components of the insulin signaling pathway are unaffected by overexpression of Sirtuin2 (FIG. 23). Thus, compounds (e.g., using methods described herein) that alter the interaction between Foxo1 and sirtuin2 or affect the ability of sirtuin2 to deacetylate Foxo1 may be compounds useful in therapy of a sirtuin2-related metabolic disorder.

### Example 3

### Diagnostic Assays

The present invention provides assays useful in the diagnosis of metabolic disorders such as diabetes and obesity, based on the discovery that sirtuin3 is downregulated in diabetes, and sirtuin2 increases adipocyte differentiation. Accordingly, diagnosis of metabolic disorders can be performed by measuring the level of expression or activity of sirtuin3 or sirtuin2 in a sample taken from a subject. This level of expression or activity can then be compared to a control sample, for example, a sample taken from a control subject, and a decrease in sirtuin3 or an increase in sirtuin2 relative to the control is taken as diagnostic of a metabolic disorder, or a risk of or propensity to a metabolic disorder.

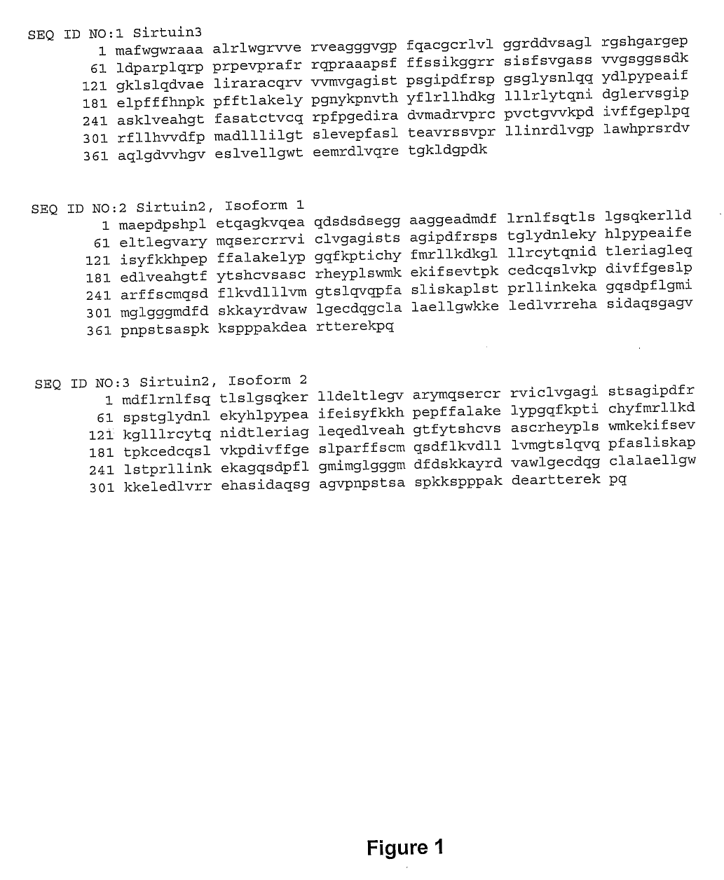

Analysis of levels of sirtuin3 or sirtuin2 mRNA or polypeptides, or activity of the polypeptides, may be used as the basis for screening the subject sample (e.g., a blood or tissue sample). Sirtuin3 and sirtuin2 nucleic acid and amino acid sequences are available in the art. For example, the nucleic acid amino acid sequences of human sirtuin3 and sirtuin2 are provided, for example, in Genbank accession numbers NM_{—}012239, NM_{—}012237, and NM_{—}030593; SEQ ID NO:1; SEQ ID NO:2; and SEQ ID NO:3 (FIG. 1). Methods for screening mRNA levels include any of those standard in the art, for example, Northern blotting. Methods for screening polypeptide levels may include immunological techniques standard in the art (e.g., western blot or ELISA), or may be performed using chromatographic or other protein purification techniques. In another embodiment, the activity (e.g., histone deactelyase activity) of sirtuin3 or sirtuin2 may be measured, where a decrease in sirtuin3 or an increase in sirtuin2 activity relative to sample taken from a control subject is diagnostic of the metabolic disorder. Such activity may be measured by any standard prior art method, for example, the method described by Yoshida et al. ((1990) J. Biol. Chem. 265, 17174-17179).

### Example 4

### Screening Methods to Identify Candidate Therapeutic Compounds

The invention also provides screening methods for the identification of compounds that bind to, or modulate expression or activity of, sirtuin3 and/or sirtuin2, that may be useful in the treatment of metabolic disorders such as diabetes or obesity. Useful compounds increase the expression or activity of sirtuin3 or decrease the expression or activity of sirtuin2.

### Screening Assays

Screening assays to identify compounds that increase the expression or activity of sirtuin3 or decrease the expression or activity of sirtuin2 (e.g., decreased binding to or deacetylation of Foxo1) are carried out by standard methods. The screening methods may involve high-throughput techniques. In addition, these screening techniques may be carried out in cultured cells or in organisms such as worms, flies, or yeast. Screening in these organisms may include the use of polynucleotides homologous to human sirtuin3 or sirtuin2. For example, a screen in yeast may include measuring the effect of candidate compounds on expression or activity of the yeast Sir2 gene (which encodes the yeast Sir2 polypeptide (SEQ ID NO:5)), or a screen in flies may include measuring the effect of candidate compounds on the expression levels or activity of the Drosophila melanogaster Sirt2 gene or Sirt2 polypeptide (SEQ ID NO:6).

Any number of methods is available for carrying out such screening assays. According to one approach, candidate compounds are added at varying concentrations to the culture medium of cells expressing a polynucleotide coding for sirtuin3 or sirtuin2. Gene expression is then measured, for example, by standard Northern blot analysis (Ausubel et al., Current Protocols in Molecular Biology, Wiley Interscience, New York, 1997), using any appropriate fragment prepared from the polynucleotide molecule as a hybridization probe. The level of gene expression in the presence of the candidate compound is compared to the level measured in a control culture medium lacking the candidate molecule. A compound which promotes an increase in sirtuin3 expression or a decrease in sirtuin2 expression is considered useful in the invention; such a molecule may be used, for example, as a therapeutic for a metabolic disorder (e.g., diabetes and obesity).

If desired, the effect of candidate compounds may, in the alternative, be measured at the level of polypeptide production using the same general approach and standard immunological techniques, such as western blotting or immunoprecipitation with an antibody specific for sirtuin3 or sirtuin2. For example, immunoassays may be used to detect or monitor the expression of sirtuin3 or sirtuin2. Polyclonal or monoclonal antibodies which are capable of binding to such a polypeptide may be used in any standard immunoassay format (e.g., ELISA, western blot, or RIA assay) to measure the level of sirtuin3 or sirtuin2. A compound which promotes an increase the expression of sirtuin3 or a decrease in the expression of the sirtuin2 is considered particularly useful. Again, such a molecule may be used, for example, as a therapeutic for a metabolic disorder (e.g., diabetes and obesity).

Alternatively, or in addition, candidate compounds may be screened for those which specifically bind to and activate sirtuin3 or inhibit sirtuin2. The efficacy of such a candidate compound is dependent upon its ability to interact with the polypeptide. Such an interaction can be readily assayed using any number of standard binding techniques and functional assays (e.g., those described in Ausubel et al., supra). For example, a candidate compound may be tested in vitro for interaction and binding with sirtuin3 or sirtuin2 and its ability to modulate its activity may be assayed by any standard assays (e.g., those described herein).

In one embodiment, candidate compounds that affect binding of sirtuin2 to Foxo1 or deacetylation of Foxo1 by sirtuin2 are identified. Disruption by a candidate compound of sirtuin2 binding to Foxo1 may be assayed using methods standard in the art. The acetylation state of Foxo1 may, for example, be assayed using an antibody to acetylated lysine (e.g., the Ack antibody), as described herein. Compounds that affect binding of sirtuin2 to Foxo1 or affect the deacetylation of Foxo1 by sirtuin2 are considered compounds useful in the invention. Such compound may be used, for example, as a therapeutic in a metabolic disorder (e.g., diabetes or obesity).

In one particular embodiment, a candidate compound that binds to sirtuin3 or sirtuin2 may be identified using a chromatography-based technique. For example, recombinant sirtuin3 or sirtuin2 may be purified by standard techniques from cells engineered to express sirtuin3 or sirtuin2 and may be immobilized on a column. A solution of candidate compounds is then passed through the column, and a compound specific for sirtuin3 or sirtuin2 is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat a metabolic disorder (e.g., diabetes and obesity). Compounds which are identified as binding to sirtuin3 or sirtuin2 with an affinity constant less than or equal to 10 mM are considered particularly useful in the invention.

Potential agonists and antagonists include organic molecules, peptides, peptide mimetics, polypeptides, and antibodies that bind to sirtuin3, sirtuin2, or a polynucleotide encoding either sirtuin3 or sirtuin2 and thereby increase or decrease its activity. Potential antagonists include small molecules that bind to and occupy the binding site of sirtuin2 thereby preventing binding of NAD⁺ or Foxo1, or preventing deacetylation of Foxo1 such that normal biological activity is prevented. Other potential antagonists include antisense molecules. Alternatively, small molecules may act as agonists and bind sirtuin3 such that its activity is increased.

Polynucleotide sequences coding for sirtuin3 or sirtuin2 may also be used in the discovery and development of compounds to treat metabolic disorders (e.g., diabetes and obesity). Sirtuin3 or sirtuin2, upon expression, can be used as a target for the screening of drugs. Additionally, the polynucleotide sequences encoding the amino terminal regions of the encoded polypeptide or Shine-Delgarno or other translation facilitating sequences of the respective mRNA can be used to construct antisense sequences to control the expression of the coding sequence of interest. Polynucleotides encoding fragments of sirtuin2 may, for example, be expressed such that RNA interference takes place, thereby reducing expression or activity of sirtuin2.

The antagonists and agonists of the invention may be employed, for instance, to treat a variety of metabolic disorders such as diabetes and obesity.

Optionally, compounds identified in any of the above-described assays may be confirmed as useful in delaying or ameliorating metabolic disorders in either standard tissue culture methods or animal models and, if successful, may be used as therapeutics for treating metabolic disorders.

Small molecules provide useful candidate therapeutics. Preferably, such molecules have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of treating a metabolic disorder (e.g., diabetes and obesity) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating metabolic disorders should be employed whenever possible.

When a crude extract is found to have an activity that increases sirtuin3 expression or activity or decreases sirtuin2 expression or activity, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating a metabolic disorder (e.g., diabetes and obesity). Methods of fractionation and purification of such heterogenous extracts are known in the art. If desired, compounds shown to be useful agents for the treatment of a metabolic disorder (e.g., diabetes and obesity) are chemically modified according to methods known in the art.

### Histone Deacetylase (HDAC) Inhibitors

Histone deacetylase inhibitors and their analogs may be used in the screening methods of the invention, particularly in screens designed to identify inhibitors of sirtuin2 activity. Histone deacetylase inhibitors are used, for example, in cancer therapy, and in the treatment of inflammation and are a group of compounds that include, for example, cyclic peptides (e.g., depsipeptides such as FK228), short chain fatty acids (e.g., phenylbutyrate and valproic acid), benzamides (e.g., CI-994 and MS-27-275), and hydroxamic acids (e.g., suberoylanilide hydroxamic acid (SAHA)) as described in Richon and O'Brien ((2002) Clin. Canc. Res. 8, 662-664). Cyclic peptides and analogs useful in the invention are described, for example, in U.S. Pat. No. 6,403,555. Short chain fatty acid HDAC inhibitors are described in, for example, U.S. Pat. Nos. 6,888,027 and 5,369,108. Benzamides analogs are described, for example, in U.S. Pat. No. 5,137,918. Analogs of SAHA are described, for example, in U.S. Pat. No. 6,511,990. Any of these compounds or other HDAC inhibitors may be used in the methods of the invention, including screening assays to identify compounds useful in the treatment of metabolic disorders (e.g., diabetes or obesity).

Any of the HDAC inhibitors described above may be chemically modified to increase binding and/or binding specificity of the HDAC inhibitor for sirtuin2 as well as to increase potency of the histone deacetylase inhibition of sirtuin2 as compared to the unmodified HDAC inhibitor. Such modifications are standard in the art and include, for example, alkylation, hydrogenation, halogenation, carboxylation, and hydroxylation.

### Example 5

### Treatment of a Metabolic Disorder

The invention also provides methods for treating metabolic disorders such as diabetes and obesity by administration of a compound that increases expression or activity of sirtuin3 or decreases expression or activity of sirtuin2 (e.g., a histone deacetylase inhibitor) in a subject. The compounds used in the treatment of metabolic disorders may, for example, be compounds identified using the screening methods described herein.

### Sirtuin3

Treatment of a subject with a metabolic disorder such as diabetes may be achieved by administration of sirtuin3. Administration may be by any route described herein; however, parenteral administration is preferred. Additionally, the sirtuin3 polypeptide administered may include modifications such as post-translational modifications (e.g., glycosylation, phosphorylation), or other chemical modifications, for example, modifications designed to alter distribution of sirtuin3 within the subject or alter rates of degradation and/or excretion of sirtuin3.

### HDAC Inhibitors

Any of the HDAC inhibitors (e.g., HDAC inhibitors described herein) may be used in the treatment methods of the invention, especially in the treatment of metabolic disorders (e.g., obesity) characterized by an increase in sirtuin2 expression or activity. Preferred HDAC inhibitors are those which preferentially inhibit a Class III NAD⁺-dependent histone deacetylase and most preferably inhibit sirtuin2 expression or activity (e.g., deacetylation of Foxo1). HDACs may be administered by any route, or in any dose, frequency, or formulation (e.g., those described herein) that achieves in vivo concentrations sufficient for treatment of a metabolic disorder.

### Dominant Negative Sirtuin2

A dominant negative sirtuin2 protein such as H232Y sirtuin2 (Dryden et al., (2003) Mol. Cell. Biol. 23, 3173-3185) may also be used in the treatment methods of the invention, especially for those characterized by an increase in sirtuin2 expression or activity. Dominant negative sirtuin2 may be administered by any route, or in any dose, frequency, or formulation (e.g., those described herein) that achieves in vivo concentrations sufficient for treatment of a metabolic disorder. Parenteral administration is preferred.

### Other Sirtuin2 Inhibitors

Sirtuin2 inhibitors that may be used in the treatment methods of the invention also include those described by Tervo et al., ((2004) J. Med. Chem. 47, 6292-6298), or modifications or derivatives thereof. Other sirtuin2 inhibitors include splitomicin, sirtinol (from Arabidopsis), and nicotinamide. Such compounds may be administered by any route, or in any dose, frequency, or formulation (e.g., those described herein) that achieves in vivo concentrations sufficient for treatment of a metabolic disorder.

Sirtuin2 inhibitors also include antibodies (for example, monoclonal antibodies) that specifically bind the sirtuin2 protein. Such antibodies may be made by any standard method and tested for their ability to block sirtuin2 activity either directly or indirectly. These antibodies may be modified in any way to make them more appropriate for human administration. For example, they may be single-chain antibodies or humanized antibodies. Again, these antibodies are administered by any route, formulation, frequency, or in any dose that achieves in vivo concentrations sufficient for treatment of a metabolic disorder.

### Gene Therapy

Increases in sirtuin3 expression or activity or decreases in sirtuin2 expression or activity may also be achieved through introduction of gene vectors into a subject. To treat a metabolic disorder such as diabetes, sirtuin3 expression may be increased, for example, by administering to a subject a vector containing a polynucleotide sequence encoding sirtuin3, operably linked to a promoter capable of driving expression in targeted cells. In another approach, a polynucleotide sequence encoding a protein that increases transcription of the sirtuin3 gene may be administered to a subject with a metabolic disorder. Any standard gene therapy vector and methodology may be employed for such administration.

Alternatively, to decrease expression of sirtuin2 for treating a metabolic disorder such as obesity, RNA interference (RNAi) may be employed. Vectors containing a target sequence, such as a short (for example, 19 base pair) sense target sequence and corresponding antisense target sequence joined by a short (for example, 9 base pair) sequence capable of forming a stem-loop structure, of the sirtuin2 mRNA transcript may be administered to a subject with a metabolic disorder. When this vector is expressed in cells, small, inhibitory RNA (siRNA) molecules are generated from this stem-loop structure, and these bind to sirtuin2 mRNA transcripts, which results in increased degradation of the targeted mRNA transcripts relative to untargeted transcripts. To test the efficacy of different sequences in mammalian cell culture systems, the pSuper RNAi System (OligoEngine, Seattle, Wash.), for example, may be employed. Preferred sequences for targeting may include those human sequences that correspond to exons 4 and 9 of the Sirt2 mouse mRNA transcript.

In another embodiment, reduction of sirtuin2 activity may be achieved through the administration to a subject of a vector containing a gene coding for a dominant negative sirtuin2 protein such as human H232Y sirtuin2 (Dryden et al., (2003) Mol. Cell. Biol. 23, 3173-3185) to treat a metabolic disorder such as obesity. Expression of this protein in the subject will reduce endogenous sirtuin2 activity, thereby treating the metabolic disorder.

### Formulation of Pharmaceutical Compositions

The administration of any compound described herein (e.g., histone deacetylase inhibitors) or identified using the methods of the invention may be by any suitable means that results in a concentration of the compound that treats a metabolic disorder. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously or intramuscularly), rectal, cutaneous, nasal, vaginal, inhalant, skin (patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

### Parenteral Compositions

The composition containing compounds described herein or identified using the methods of the invention may be administered parenterally by injection, infusion, or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in the form of aqueous suspensions, microspheres, microcapsules, magnetic microspheres, oil solutions, oil suspensions, or emulsions. The composition may also be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine), poly(lactic acid), polyglycolic acid, and mixtures thereof. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)) or combinations thereof.

### Solid Dosage Forms for Oral Use

Formulations for oral use include tablets containing the active ingredient(s) in a mixture with non-toxic pharmaceutically acceptable excipients, and such formulations are known to the skilled artisan (e.g., U.S. Pat. Nos. 5,817,307, 5,824,300, 5,830,456, 5,846,526, 5,882,640, 5,910,304, 6,036,949, 6,036,949, 6,372,218, hereby incorporated by reference). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and anti-adhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the compound in a predetermined pattern (e.g., in order to achieve a controlled release formulation) or it may be adapted not to release the agent(s) until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols, and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate, may be employed.

The solid tablet compositions may include a coating adapted to protect the composition from unwanted chemical changes, (e.g., chemical degradation prior to the release of the active substances). The coating may be applied on the solid dosage form in a similar manner as that described in Encyclopedia of Pharmaceutical Technology, supra.

The compositions of the invention may be mixed together in the tablet, or may be partitioned. In one example, a first agent is contained on the inside of the tablet, and a second agent is on the outside, such that a substantial portion of the second agent is released prior to the release of the first agent.

Formulations for oral use may also be presented as chewable tablets, of as hard gelatin capsules wherein the active ingredient is mixed with an inert solid diluent (e.g., potato starch, lactose, microcrystalline cellulose, calcium carbonate, calcium phosphate, or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus, or spray drying equipment.

### Controlled Release Oral Dosage Forms

Controlled release compositions for oral use may, e.g., be constructed to release the compound by controlling the dissolution and/or the diffusion of the compound.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, DL-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax, and stearyl alcohol, carbopol 934, silicone, glyceryl tristearate, methyl acrylate-methyl methacrylate, polyvinyl chloride, polyethylene, and/or halogenated fluorocarbon.

A controlled release composition containing compounds described herein or identified using methods of the invention may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the composition with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Dosages

The dosage of any compound described herein or identified using the methods described herein depends on several factors, including: the administration method, the metabolic disorder to be treated, the severity of the metabolic disorder, whether the metabolic disorder is to be treated or prevented, and the age, weight, and health of the subject to be treated.

With respect to the treatment methods of the invention, it is not intended that the administration of a compound to a subject be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to treat hepatitis. The compound may be administered to the subject in a single dose or in multiple doses. For example, a compound described herein or identified using screening methods of the invention may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient activity in the treatment of a metabolic disorder (e.g., diabetes or obesity). Conversely, the dosage of the compound can be decreased if the metabolic disorder is reduced or eliminated.

While the attending physician ultimately will decide the appropriate amount and dosage regimen, a therapeutically effective amount of a compound described herein (e.g., histone deacetylase inhibitors) or identified using the screening methods of the invention, may be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound may be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

All patents, patent applications, and publications mentioned in this specification are herein incorporated by reference to the same extent as if each independent patent, patent application, or publication was specifically and individually indicated to be incorporated by reference.

What is claimed is:

1. A method of diagnosing a metabolic disorder, or a propensity thereto, in a subject, said method comprising analyzing the level of sirtuin3 expression or activity in a sample isolated from said subject, wherein a decreased level of sirtuin3 expression or activity in said sample relative to the level in a control sample indicates that said subject has said metabolic disorder, or a propensity thereto.

2. The method of claim 1, wherein said analyzing comprises measuring the amount of sirtuin3 RNA or protein in said sample.

3. The method of claim 1, wherein said analyzing comprises measuring the histone deacetylase activity of sirtuin3 in said sample.

4. The method of claim 1, wherein said metabolic disorder is diabetes.

5. The method of claim 1, wherein said subject is a human.

6. A method of identifying a candidate compound useful for treating a metabolic disorder in a subject, said method comprising:
(a) contacting a sirtuin3 protein with a compound; and
(b) measuring the activity of said sirtuin3, wherein an increase in sirtuin3 activity in the presence of said compound relative to sirtuin3 activity in the absence of said compound identifies said compound as a candidate compound for treating a metabolic disorder in a subject.

7. The method of claim 6, wherein said compound is selected from a chemical library.

8. The method of claim 6, wherein said sirtuin3 protein is human sirtuin3 protein.

9. The method of claim 6, wherein said method is performed in a cell.

10. The method of claim 6, wherein said method is performed in vitro.

11. The method of claim 6, wherein said metabolic disorder is diabetes.

12. A method of identifying a candidate compound useful for treating a metabolic disorder in a subject, said method comprising:
(a) contacting a sirtuin3 protein with a compound; and
(b) measuring the binding of said compound to sirtuin3, wherein specific binding of said compound to said sirtuin3 protein identifies said compound as a candidate compound for treating a metabolic disorder in a subject.

13. The method of claim 12, wherein said compound is selected from a chemical library.

14. The method of claim 12, wherein said sirtuin3 protein is human sirtuin3 protein.

15. The method of claim 12, wherein said metabolic disorder is diabetes.

16. A method of identifying a candidate compound useful for treating a metabolic disorder in a subject, said method comprising:
(a) contacting a cell or cell extract comprising a polynucleotide encoding sirtuin3 with a compound; and
(b) measuring the level of sirtuin3 expression in said cell or cell extract, wherein an increased level of sirtuin3 expression in the presence of said compound relative to the level in the absence of said compound identifies said compound as a candidate compound for treating a metabolic disorder in a subject.

17. The method of claim 16, wherein said compound is selected from a chemical library.

18. The method of claim 16, wherein said sirtuin3 is human sirtuin3.

19. The method of claim 16, wherein said metabolic disorder is diabetes.

20. A method of treating a metabolic disorder in a subject, said method comprising administering to said subject a therapeutically effective amount of a composition that increases sirtuin3 expression or activity.

21. The method of claim 20, wherein said composition comprises sirtuin3 protein.

22. The method of claim 20, wherein said composition comprises a polynucleotide encoding sirtuin3 protein.

23. The method of claim 20, wherein said metabolic disorder is diabetes.

24. The method of claim 20, wherein said subject is a human.

25. The method of claim 20, wherein said sirtuin3 is human sirtuin3.

26. A kit for treating a metabolic disorder in a subject, said kit comprising:
(a) a composition that increases sirtuin3 expression or activity; and
(b) instructions for administering said composition to a subject with a metabolic disorder.

27. A method of diagnosing a metabolic disorder, or a propensity thereto, in a subject, said method comprising analyzing the level of sirtuin2 expression or activity in a sample isolated from said subject, wherein an increased level of sirtuin2 expression or activity in said sample relative to the level in a control sample indicates that said subject has said metabolic disorder, or a propensity thereto.

28. The method of claim 27, wherein said analyzing comprises measuring the amount of sirtuin2 RNA or protein in said sample.

29. The method of claim 27, wherein said analyzing comprises measuring the histone deacetylase activity of sirtuin2 in said sample.

30. The method of claim 27, wherein said metabolic disorder is obesity.

31. The method of claim 27, wherein said subject is a human.

32. A method of identifying a candidate compound useful for treating a metabolic disorder in a subject, said method comprising:
(a) contacting a sirtuin2 protein with a compound; and
(b) measuring the activity of said sirtuin2, wherein a decrease in sirtuin2 activity in the presence of said compound relative to sirtuin2 activity in the absence of said compound identifies said compound as a candidate compound for treating a metabolic disorder in a subject.

33. The method of claim 32, wherein said compound is selected from a chemical library.

34. The method of claim 32, wherein said sirtuin2 protein is human sirtuin2 protein.

35. The method of claim 32, wherein said method is performed in a cell.

36. The method of claim 32, wherein said method is performed in vitro.

37. The method of claim 32, wherein said metabolic disorder is obesity.

38. A method of identifying a candidate compound useful for treating a metabolic disorder in a subject, said method comprising:
(a) contacting a sirtuin2 protein with a compound; and
(b) measuring the binding of said compound to sirtuin2, wherein specific binding of said compound to said sirtuin2 protein identifies said compound as a candidate compound for treating a metabolic disorder in a subject.

39. The method of claim 38, wherein said compound is selected from a chemical library.

40. The method of claim 38, wherein said sirtuin2 protein is human sirtuin2 protein.

41. The method of claim 38, wherein said metabolic disorder is obesity.

42. A method of identifying a candidate compound useful for treating a metabolic disorder in a subject, said method comprising:
(a) contacting a cell or cell extract comprising a polynucleotide encoding sirtuin2 with a compound; and
(b) measuring the level of sirtuin2 expression in said cell or cell extract, wherein a decreased level of sirtuin2 expression in the presence of said compound relative to the level in the absence of said compound identifies said compound as a candidate compound for treating a metabolic disorder in a subject.

43. The method of claim 42, wherein said candidate compound is selected from a chemical library.

44. The method of claim 42, wherein said sirtuin2 is human sirtuin2.

45. The method of claim 42, wherein said metabolic disorder is obesity.

46. A method of treating a metabolic disorder in a subject, said method comprising administering to said subject a therapeutically effective amount of a composition that decreases sirtuin2 expression or activity.

47. The method of claim 46, wherein said composition comprises an RNA that interferes with the mRNA coding for the sirtuin2 protein.

48. The method of claim 46, wherein said composition comprises a histone deacetylase inhibitor.

49. The method of claim 46, wherein said composition comprises a dominant negative sirtuin2 protein.

50. The method of claim 49, wherein said dominant negative sirtuin2 is human H232Y sirtuin2.

51. The method of claim 46, wherein said composition comprises an antibody that specifically binds sirtuin2, or is a sirtuin2-binding fragment thereof.

52. The method of claim 46, wherein said metabolic disorder is obesity.

53. The method of claim 46, wherein said subject is a human.

54. A kit for treating a subject with a metabolic disorder, said kit comprising:
(a) a composition that decreases sirtuin2 expression or activity; and
(b) instructions for administering said composition to a subject with a metabolic disorder.
